(19) United States
(12) Patent Application Publication
(10) Pub. No.: US 2002/0009713 A1
(43) Pub. Date: Jan. 24, 2002

(54) Methods for identifying modulators of neuronal growth

(76) Inventors: Freda D. Miller, Montreal (CA); Andrew Vaillant, Montreal (CA)

Correspondence Address: CLARK & ELBING LLP, 176 FEDERAL STREET, BOSTON, MA, 02110-2214, US

(21) Appl. No.: 09/852,512

(22) Filed: May 10, 2001

Related U.S. Application Data
(60) Provisional application No. 60/203,560, filed on May 11, 2000.

(51) Int. Cl. C12Q 1/00; C12Q 1/68; C12N 5/08

(52) U.S. Cl. 435/4; 435/6; 435/368

(57) ABSTRACT

The invention features methods identifying compounds that modulate neuronal growth. The invention also features methods of modulating neuronal growth by modulating the p75^{NTR} or MEK/MAPK pathways, and methods of identifying compounds that do the same.

### CROSS-REFERENCE TO RELATED APPLICATIONS

This application claims benefit from U.S. Provisional Application Serial No. 60/203,560, filed May 11, 2000 (now pending), which is hereby incorporated by reference.

### BACKGROUND OF THE INVENTION

The invention relates to neuronal growth.

The development of strategies to promote repair of the degenerating or traumatized nervous system is a major ongoing therapeutic challenge. One approach to the problem of nerve trauma and regeneration is the development of relevant therapeutics that can promote the survival or repair of traumatized neurons. One of the fundamental technological problems associated with the identification of such therapies, however, is the lack of suitable high throughput screens for compounds effective on primary neurons. This lack of suitable screens derives from two major considerations. First, recent findings indicate that primary neurons differ significantly in their survival and growth signaling pathways from any of the transformed or immortalized cell lines that are currently available, making screens using cell lines unreliable. Second, postmitotic neurons are (i) only available in relatively small amounts; (ii) difficult to genetically manipulate; and (iii) difficult to culture as a purified cell population.

Neurons within the peripheral nervous system, including those affected by peripheral neuropathy, have the capacity for axonal growth and regeneration. Moreover, under certain conditions, peripheral neurons are capable of axonal growth and regeneration following axonal injury. It is not well understood what regulates such axonal growth and regeneration. It would be desirable to stimulate neuronal growth for the treatment of a neurodegenerative disease or a neurotrauma. To this end, it would also be desirable to more fully understand the signals that regulate neuronal growth.

### SUMMARY OF THE INVENTION

The invention features methods for identifying compounds for the modulation of neuronal cell growth, including regeneration. The invention also features a method and reagents for modulatingneuronal growth. The method is based upon our discovery that p75^{NTR} and Trk-mediated MEK/MAPK signaling modulate neuronal growth but not survival.

Accordingly, in a first aspect, the invention features a method for identifying a compound that increases or decreases neuronal growth. The method includes the steps of: (a) contacting a culture that includes neurons with a test compound; and (b) measuring the amount of a protein or RNA that is preferentially found in the axons, dendrites, or neurites of the neurons. An increase in the amount of the protein or RNA relative to the amount of the protein or RNA in a culture not contacted with the test compound identifies the test compound as a compound that increases neuronal growth. Conversely, a decrease in the amount of the protein or RNA relative to the amount of the protein or RNA in a culture not contacted with the test compound identifies the test compound as a compound that decreases neuronal growth.

Exemplary proteins that are preferentially found in neurites include alpha-tubulin, beta-tubulin, GAP-43, neurofilament light chain, neurofilament medium chain, neurofilament heavy chain, microtubule-associated protein (MAP) 1B, MAP4, MAP2C, and MAP2D. Proteins preferentially found in dendrites include, for example, MAP2A, MAP2B, dendrin, type II calcium-calmodulin-dependent protein (CamIIK), metabotropic glutamate receptor (mGluR) 1A, mGluR2, and type 1A serotonin receptor (5-HT1a), while RNAs preferentially found in dendrites include MAP2A RNA, MAP2B RNA, dendrin RNA, and CamIIK RNA. Examples of proteins preferentially found in axons include phosphorylated MAP1B, phosphorylated neurofilament heavy chain, and type 1B serotonin receptor.

In another aspect, the invention features a method of identifying a compound that increases neuronal growth, the method including: (a) contacting a neuron with a neurotrophin at levels sufficient to support cell survival; (b) contacting the neuron with a neuronal growth-inhibiting compound; (c) contacting the neuron with a test compound; and (d) assaying the level of neuronal growth in the neuron, wherein an increase in neuronal growth relative to a neuron contacted with the NGF and the neuronal growth-inhibiting compound, but not contacted with the test compound, identifies the test compound as a compound that increases neuronal growth.

It is desirable that the foregoing method be quantifiable. In one example, the level of neuronal growth is assayed by measuring the amount of total protein in a culture or by measuring the level of a protein or mRNA that is preferentially found in dendrites, axons, or both (i.e., neurites).

The neurotrophin can be, for example, NGF, BDNF, NT-3, or NT-4/5. The neuronal growth-inhibiting compound can be, for example, an inhibitor of MEK signaling. Such inhibitors are known in the art (e.g., PD98059 (Pang et al., J. Biol. Chem. 270:13585-13588,1995); U0126 (Duncia et al., Bioorg. Med. Chem. Lett. 8:2839-2844, 1998)). The neuronal growth-inhibiting compound can also be an activator of p75^{NTR} signaling. If the neuron does not have TrkB receptors, the growth-inhibiting compound can be BDNF or a BDNF analog.

In a related aspect, the invention features another method of identifying a compound that increases neuronal growth. This method includes (a) inhibiting MEK signaling in a neuron; (b) contacting the neuron with a test compound; and (c) assaying the level of neuronal growth in the neuron, wherein an increase in neuronal growth relative to a neuron in which MEK signaling is inhibited but which is not contacted with the test compound identifies the test compound as a compound that increases neuronal growth.

MEK signaling can be inhibited, for example, by expression of a dominant-negative MEK in the neuron, or by contacting the neuron with an inhibitor of MEK signaling (e.g., U0126 or PD98059). It is desirable that MEK signaling is inhibited by at least 25%, by at least 50%, or even by at least 75%.

In a related aspect, the invention features another method of identifying a compound that increases neuronal growth, the method including (a) contacting a neuron with a test compound; and (b) assaying the level of MEK signaling in the neuron, wherein an increase in MEK signaling relative to a neuron not contacted with the test compound identifies the test compound as a compound that increases neuronal growth.

In still another aspect, the invention features another method of identifying a compound that increases neuronal growth. This method includes (a) activating p75^{NTR} signaling in a neuron; (b) contacting the neuron with a test compound; and (c) assaying the level of neuronal growth in the neuron, wherein an increase in neuronal growth relative to a neuron in which p75^{NTR} signaling is activated but which is not contacted with the test compound identifies the test compound as a compound that increases neuronal growth.

p75^{NTR} signaling can be activated, for example, by introducing ceramide into the neuron. Preferably, p75^{NTR} signaling is activated by at least 25%, more preferably by at least 50%, and most preferably by at least 75%.

In a related aspect, the invention features a method of identifying a compound that increases neuronal growth, the method including (a) contacting a neuron with a test compound; and (b) assaying the level of p75^{NTR} signaling in the neuron, wherein a decrease in p75^{NTR} signaling relative to a neuron not contacted with the test compound identifies the test compound as a compound that increases neuronal growth.

The neuron can be, for example, a sympathetic neuron, a cholinergic neuron, a sensory neuron, a cortical neuron, a motor neuron, or a neuron derived from the dorsal root ganglia. It is desirable that the neuron is from a mammal (e.g., a human). The neuron can be in vitro or in vivo.

The test compound can be selected from the group consisting of a peptide or protein, a carbohydrate, a lipid, a nucleic acid molecule, a natural organic molecule, a synthetically derived organic molecule, an antibody (e.g., one that binds p75^{NTR}), or a derivative or modification thereof.

In yet another aspect, the invention a method of treating a patient having a neurodegenerative disease, the method including administering to the patient a compound that increases MEK signaling in a neuron in the patient.

In a related aspect, the invention features a method of treating a patient having a neurodegenerative disease, the method including administering to the patient a compound that decreases p75^{NTR} signaling in a neuron in the patient.

By “neuron” is meant a cell of embryonic ectodermal origin derived from any part of the nervous system of an animal. Neurons express well-characterized neuron-specific markers, including class III β-tubulin, MAP2, and neurofilament proteins. Neurons include, without limitation, cortical neurons, motor neurons, sensory neurons, cholinergic neurons, sympathetic neurons, and neurons derived from the dorsal root ganglion. Also considered to be a neuron is a cell expressing one or more neuron-specific markers, regardless of the origin. For example, neurons can be derived from cell lines (e.g., PC-12 cells, P-19 cells, and neuroblastoma cells) and stem cells (e.g., embryonic stem cells, mesenchymal stem cells, neural stem cells).

By “neuronal growth” is meant an increase in process network density (e.g., axonal, dendritic, or neuritic growth, including branching), cell migration, or target innervation. Any of these aspect of neuronal growth may be measured using techniques known in the art and as described herein.

By “neuronal processes” is meant the axons, dendrites, or neurites (axons and dendrites) formed by a neuron. These are routinely characterized (e.g., in terms of shape, number, and targets innervated) using, for example, standard microscopic techniques.

By “NGF” is meant nerve growth factor which, in general, binds the TrkA receptor and promotes neuronal survival and growth and may be purified from, e.g., mouse salivary glands or produced recombinantly.

By “BDNF” is meant brain-derived neurotrophic factor, which, in general, binds the p75^{NTR} neurotrophin receptor and inhibits neuronal growth and target innervation, or binds TrkB and promotes neuronal growth and survival.

By “test compound” is meant a chemical, be it naturally-occurring or artificial, that is surveyed for its ability to modulate cell death, by employing one of the assay methods described herein. Test compounds may include, for example, peptides, polypeptides, antibodies (and fragments thereof), synthesized organic molecules, naturally occurring organic molecules, nucleic acid molecules, and components or derivatives thereof.

By “assaying” is meant analyzing the effect of a treatment, be it chemical or physical, administered to whole animals or cells derived therefrom. The material being analyzed may be an animal, a cell, a lysate or extract derived from a cell, or a molecule derived from a cell. The analysis may be, for example, for the purpose of detecting altered gene expression, altered RNA stability, altered protein stability, altered protein levels, altered protein phosphorylation, or altered protein biological activity. The means for analyzing may include, for example, antibody labeling, immunoprecipitation, phosphorylation assays, and methods known to those skilled in the art for detecting nucleic acids.

By “modulating” is meant changing, either by decrease or increase.

By “p75^{NTR} pathway” is meant the entire signal transduction pathway, or part thereof, that is sufficient to transfer a signal that inhibits neuronal growth in the presence of NGF. Components of the p75^{NTR} pathway that can contribute to the positive regulation of this pathway (i.e., repress neuronal growth) are, for example, activated MEKK, activated JNK, dominant negative Ras (e.g., N17Ras), dominant negative Akt, c-jun, p53, p21, and Bax.

By “Trk” is meant TrlA, TrkB, or TrkC.

By “inhibitor of MEK/MAPK pathway” is meant any compound that inhibits MEK/MAPK-mediated neuronal growth.

By “alteration in the level of gene expression” is meant a change in gene activity such that the amount of a product of the gene, i.e., mRNA or polypeptide, is increased or decreased, or that the stability of the mRNA or the polypeptide is increased or decreased.

By “reporter gene” is meant any gene which encodes a product whose expression is detectable and/or quantifiable by physical, immunological, chemical, biochemical or biological assays. A reporter gene product may, for example, have one of the following attributes, without restriction: a specific nucleic acid/chip hybridization pattern, fluorescence (e.g., green fluorescent protein), enzymatic activity (e.g., lacZ/β-galactosidase, luciferase, chloramphenicol acetyltransferase), toxicity (e.g., ricin A), or an ability to be specifically bound by a second molecule (e.g., biotin or a detectably labelled antibody). It is understood that any engineered variants of reporter genes, which are readily available to one skilled in the art, are also included, without restriction, in the foregoing definition.

By “operably linked” is meant that a gene and a regulatory sequence are connected in such a way as to permit expression of the gene product under the control of the regulatory sequence.

By a “transgene” is meant a nucleic acid sequence which is inserted by artifice into a cell and becomes a part of the genome of that cell and its progeny. Such a transgene may be partly or entirely heterologous to the cell, or may be in a position or under regulatory control which is distinct from that of a naturally occurring cell.

By “transgenic animal” is meant an animal having a transgene as described above.

By “increase in neuronal growth” is meant an increase in the number, length, or branching of axons, dendrites, or neurites of a neuron compared to a control neuron. The neuron may also exhibit other aspects of neuronal growth as described above.

By a protein or mRNA that is “preferentially found” in dendrites is meant one that is present in a substantially greater amount in dendrites than it is in axons. Abundance of the protein or mRNA can be at least 5-fold greater in dendrites than it is in axons (per unit length), or even 10-fold more abundant (or greater) in dendrites than it is in axons. Similarly, a protein or mRNA that is “preferentially found” in axons is one that is in a substantially greater amount in axons than it is in dendrites, preferably at least 5-fold and more preferably at least 10-fold greater. A protein or mRNA that is “preferentially found” in neurites is one that is in substantially greater amount in axons and/or dendrites than it is in the cell soma. Again, it is desirable that the difference be at least 5-fold, at least 10-fold, or greater.

The methods described herein allow for the identification of a compound that stimulates neuronal growth. Moreover, these methods are readily adapted for high throughput drug screening.

Other features and advantages of the invention will be apparent from the following description of the preferred embodiments thereof and from the claims.

### BRIEF DESCRIPTION OF THE DRAWINGS

FIGS. 1A-1C are schematic illustrations that show that BDNF signaling through p75^{NTR} decreases the growth of sympathetic neurons in vitro without affecting their survival. FIG. 1A shows results of colorimetric MTT assays to measure mitochondrial function and cell survival. FIGS. 1B and 1C show quantitative analysis of neurite process density in sympathetic neuron cultures grown in the presence of NGF or NGF plus BDNF.

FIGS. 2A-2D are photographs that show that exogenous BDNF inhibits and anti-BDNF and anti-p75^{NTR} each enhance NGF-promoted growth of sympathetic neurons in vitro.

FIG. 3 is a photograph of an immunoblot that shows that a function-blocking BDNF antibody neutralizes the ability of exogenous BDNF to activate TrkB.

FIGS. 4A-4F are schematic illustrations that show that function-blocking antibodies directed against BDNF or p75^{NTR} enhance growth of sympathetic neurons without affecting their survival.

FIG. 4A shows results of colorimetric MTT assays to measure mitochondrial function and cell survival.

FIGS. 4B-4F depict quantitative analysis of neuritic process density in sympathetic neuron cultures grown in the presence of NGF, NGF plus anti-BDNF (FIGS. 4B and 4C), NGF plus anti-p75^{NTR} (FIGS. 4D and 4E), or increased NGF (FIG. 4F).

FIGS. 5A-5D are schematic illustrations that depict an analysis of neurite outgrowth in response to NGF or NGF plus BDNF in p75^{NTR-/-} versus p75^{NTR+/+} sympathetic neurons.

FIGS. 5A and 5B show results of colorimetric MTT assays to measure mitochondrial function and survival of murine sympathetic neurons in response to NGF or NGF plus BDNF.

FIGS. 5C-5D show quantitative analysis of neuritic process density in p75^{NTR-/-} versus wild-type murine sympathetic neurons in response to NGF or NGF plus BDNF.

FIGS. 6A-6D are photographs that show that p75^{-/-} sympathetic neurons show enhanced neuritogenesis in response to NGF, but do not respond to exogenous BDNF. The phase contrast micrographs are of cultured Coomassie Blue-stained wild-type (FIGS. 6A-6B) and p75^{NTR}^{-/-} murine sympathetic neurons (FIGS. 6C-6D) maintained in 50 ng/ml NGF for two days, and then switched to 7.5 ng/ml NGF (FIGS. 6A and 6C), or 7.5 ng/ml NGF plus 100 ng/ml BDNF (FIGS. 6B and 6D).

FIGS. 7A-7E are photographs of immunoblots that show BDNF and p75^{NTR} levels in transgenic mice and that BDNF and p75^{NTR} are present in the pineal gland during the period of sympathetic target innervation.

FIG. 7A represents immunoblot analysis for BDNF in the adult rat pineal gland.

FIG. 7B represents an immunoblot analysis for BDNF in the pineal gland of BDNF^{-/-}, BDNF^{+/-}, and BDNF^{+/+} litter mates at P13 to P15.

FIG. 7C represents an immunoblot analysis for p75^{NTR} in the developing postnatal rat pineal gland.

FIGS. 7D and 7E show that levels of tyrosine hydroxylase, a specific marker for sympathetic fibers, are increased in the pineal glands of BDNF^{+/-} and BDNF^{-/-} mice.

FIG. 7D shows an immunoblot analysis of tyrosine hydroxylase, a specific marker for sympathetic fibers, in equal amounts of protein from the pineal glands of BDNF^{+/+}, BDNF^{+/-}, and BDNF^{-/-} litter mates at P13 to P15.

FIG. 7E shows an immunoblot analysis of p75^{NTR} in equal amounts of protein from the pineal glands of BDNF^{+/+}, BDNF^{+/-}, and BDNF^{-/-} litter mates at P13 to P15.

FIGS. 8A-8J are photographs that show that the pineal gland is hyperinnervated with sympathetic fibers in BDNF^{-/-} mice at P13. The photographs are of immunohistochemical analysis for tyrosine hydroxylase, a specific marker for sympathetic fibers, in sections of the pineal gland from a control litter mate (FIGS. 8A, 8C-8E) relative to a BDNF^{-/-} litter mate (FIGS. 8B, 8F-8H).

FIG. 9A is a schematic illustration of MEK isoforms. DN MEK is a dominant-negative MEK isoform, while G1C (ACT MEK) is constitutively active. Also shown is a schematic illustration of an adenoviral vector carrying MEK and GFP.

FIG. 9B is a series of photographs of immunoblots showing that adenovirally expressed DN MEK decreased MAPK phosphorylation in both PC 12 cells and SCG neurons.

FIG. 9C is a photograph of immunocytochemical detection of adenovirally infected cells expressing DN MEK.

FIG. 9D is a schematic illustration showing that none of the MEK isoforms increased SCG neuronal survival in the absence of NGF.

FIG. 10A is a schematic illustration of a chamber used in neuronal growth assays.

FIG. 10B is a series of photographs showing tubulin staining of hepatocyte growth factor (HGF)-induced neurites from DN MEK-expressing neurons (right panel) or control neurons (left panel).

FIG. 10C is a quantification of the neuritic growth exemplified in FIG. 10B.

FIG. 11 is a photograph showing the expression pattern of reporter gene operably linked to the Tα1 α-tubulin promoter.

FIG. 12 is a schematic illustration showing that in vivo expression of DN MEK decreases P-MAPK levels in the hippocampus.

FIGS. 13A and 13B are schematic illustrations showing that expression of DN MEK decreases innervation of the pineal gland by sympathetic neurons by approximately 75% (FIG. 13A) without a reduction in the number of SCG neurons (FIG. 13B).

FIGS. 14A and 14B are a series of photographs and a schematic, respectively, showing results with DN MEK.

FIG. 14A is a photograph showing the medial branch of the dorsal cutaneous nerve having selective loss of the unmyelinated C-fibers in mice expressing DN MEK (left), relative to the control (right).

FIG. 14B is a schematic illustration showing that expression of DN MEK results in a loss of C-fibers in the dorsal cutaneous nerve.

FIG. 15 is a schematic illustration showing that MEK inhibitor U0126 blocks NGF/TrkA-mediated neurite outgrowth in primary sympathetic neurons.

FIG. 16 is a schematic illustration showing that U0126 blocks BDNF/TrkB-mediated neurite outgrowth in primary sympathetic neurons that were made to exogenously express TrkB by infection with a recombinant adenovirus.

FIGS. 17A and 17B are schematic illustrations showing that a mutant TrkB receptor, exhibiting reduced activation of MEK, is severely deficient in promoting neurite outgrowth.

FIG. 18 is a schematic illustration showing that inhibition of MEK activity by overexpression of DN MEK attenuated TrkB-mediated neurite outgrowth in primary sympathetic neurons made to exogenously express TrkB by infection with a recombinant adenovirus.

FIGS. 19A-19C are photographs showing HMW-MAP2 localization following treatment for three days with 10 ng/ml NGF (FIG. 19A), 50 mM KCl (FIG. 19B), or 10 ng/ml NGF plus 50 mM KCl (FIG. 19C). Scale bar=100 μM.

FIGS. 20A and 20B are high magnification photogrpahs of MAP2 staining of SCG neurons in the presence of NGF (FIG. 20A) and NGF+KCl (FIG. 20B). Scale bar=50 μM.

FIG. 21 is a schematic illustration showing image analysis of cultures shown in FIGS. 19A-19C. Lengths of individuals dendrites were counted and histogram analysis was performed using bin sizes of 2 μM. The histogram analyses were then normalized so that the population data could be compared between different treatments.

FIG. 22 is a schematic illustration showing immuno-dot blot analysis of MAP2 association and tubulin dynamics in SCG neurons. The relative amounts of MAP2 present per μg of assembled tubulin (relative association of MAP2 with microtubules) were determined by the ratio of AP20 signal (dendrite specificMAP2) to DM1A signal (polymerized tubulin) following three days of treatment with 10 ng/ml NGF, 50 mM KCl, or 10 ng/ml NGF plus 50 mM KCl.

FIGS. 23A-23D are a series of photographs showing MAP2 immunofluorescence in SCG neurons treated for three days with 50 mM KCl (FIG. 23A), followed by two days treatment with 10 ng/ml NGF alone (FIG. 23B) or 10 ng/ml NGF plus 50 mM KCl (FIG. 23C) followed by two days treatment with 10 ng/ml NGF alone (FIG. 23D). Scale bar=100 μM.

FIGS. 24A-24J are a series of photographs showing MAP2 immunofluorescence in SCG neurons treated for three days with 10 ng/ml NGF (FIGS. 24A-24E) or 10 ng/ml NGF plus 50 mM KCl (FIGS. 24F-24J). Cultures were simultaneously exposed to 10 μM CaMKII inhibitor KN-62 (FIG. 24B, 24G), 50 μM MEK inhibitor PD98059 (FIGS. 24C, 24H), 100 nM PKA inhibitor H-89 (FIGS. 24D, 24I) or 10 μM adenylate cyclase inhibitor Forskolin (FIGS. 24E, 24J). Control cultures are shown in FIGS. 24A and 24F. Scale bar 100 μM.

FIG. 25 is a schematic illustration showing image analysis of MAP2 localization in the SCG cultures shown in FIGS. 24A-24J. Note that inhibitation of CaMKII or MEK activities resulted in a suppression of dendritic maturation in response to KCl.

FIG. 26 is a schematic illustration showing immuno-dot blot analysis of MAP2 association and microtubule dynamics in the SCG cultures shown in FIGS. 24A-24J. The data represent the effect of kinase inhibitors on the relative amounts of MAP2 associated per μg of assembled tubulin.

FIG. 27 is a schematic illustration showing biochemical analysis of the effects of NGF, KCl, and kinase inhibitors on MAP2 levels. Note both KN-62 and PD98059 reduce MAP2 levels.

### DETAILED DESCRIPTION

We have discovered that Trk and p75^{NTR} have functionally antagonistic actions on neuron growth and target innervation, with NGF acting via TrkA to promote growth and BDNF via p75^{NTR} to inhibit growth. Specifically, in rat sympathetic neurons (which do not express TrkB), BDNF signals through p75^{NTR} to inhibit neuronal growth. Similarly, function-blocking BDNF antibodies can enhance neuronal growth of these cells. Both exogenous and autocrine BDNF mediate this effect via p75^{NTR} since (i) BDNF does not inhibit growth of neurons lacking p75^{NTR}; (ii) function-blocking p75^{NTR} antibodies enhance NGF-mediated growth, and; (iii) p75^{NTR-/-} sympathetic neurons grow better in response to NGF than do their wild-type counterparts. Thus, BDNF, made by sympathetic neurons and/or their target organs, acts via p75^{NTR} to antagonize NGF-mediated growth and target innervation, indicating that sympathetic target innervation is determined by the balance of positively- and negatively-acting neurotrophins present in developing, and potentially, mature targets.

We have also discovered that two kinases, MAP kinase (MAPK) and MAP kinase kinase (MEK), transduce Trk-mediated neuronal growth signals, but are not required for NGF-mediated cell survival. Thus, any compound that blocks p75^{NTR} signaling or promotes MEK/MAPK signaling is expected to promote neuronal growth of sympathetic neurons. Accordingly, we have developed assays in which neuronal growth is blocked either by the activation of the p75^{NTR} pathway, or by disruption of MEK/MAPK signaling. These assays can be employed to identify compounds that overcome the signals inhibiting neuronal growth and thus stimulate neuronal growth and target innervation. The identified compounds are likely to be genetically downstream of both p75 and MEK/MAPK and, thus, are expected to be selective for promoting neuronal growth. Compounds identified using the screens described herein are useful for treating neurodegenerative disease (e.g., Alzheimer's disease, Huntington's disease, Parkinson's disease, and amyotrophic lateral sclerosis) or disease resulting from trauma, such as ischemic stroke or axotomy.

We believe this functional antagonism between Trk-mediated MEK signaling and p75^{NTR} signaling may be generalized to neurons other than sympathetic neurons. Evidence for this is found in p75^{NTR-/-} mice, in which the number of basal forebrain cholinergic neurons increases and the hippocampus is hyperinnervated, a phenotype reminiscent of what is seen for sympathetic neurons in p75^{NTR-/-} and BDNF^{-/-} mice. Moreover, this same functional antagonism may also occur in adult sensory neurons, as well as in neurons of the dorsal root ganglia.

Without wishing to be bound to a particular theory, we believe that p75^{NTR} inhibits Trk-mediated neuronal growth through p75^{NTR}-mediated generation of intracellular ceramide. Thus, activation of p75^{NTR} could well attenuate neurite growth via increased ceramide.

We have also discovered that inhibition of MEK signaling (e.g., through the expression of a dominant-negative MEK (DN MEK) or the administration of U0126, a specific inhibitor of MEK) interferes with Trk-mediated cell growth without perturbing cell survival. For example, transgenic animals expressing DN MEK exhibit a decrease of neuronal growth in the pineal gland. Thus, activation of p75^{NTR} and suppression of MEK signaling produce a similar phenotype.

The present discoveries can be exploited in useful high-throughput screening assays for compounds that stimulate neuronal growth and target innervation. Development of assays for compounds which modulate neuronal growth and target innervation with high specificity requires, first, identification of key, specific steps in the neuronal growth pathway and, second, development of assays to measure changes at these steps. To determine the mechanism whereby the neurotrophins stimulate the survival and growth of normal and injured neurons, we have asked whether specific components of the p75^{NTR} and MEK/MAPK pathways play a role in modulating neuronal growth, focusing our studies on cultured rat sympathetic cervical ganglion (SCG) neurons in order to determine the exact nature of this regulation. In addition, we have taken advantage of transgenic mice lacking either BDNF or p75^{NTR} or, alternatively, expressing a dominant negative MEK to demonstrate the role these factors play in vivo. The examples below show that alterations in the signaling of each of these pathways alters neuronal growth. These findings serve as a basis for the development of assays for the identification of compounds that modulate one or both signaling pathways and, thus, modulate neuronal growth.

Primary Screens for Compounds that Modulate the BDNF/p75^{NTR} or MEK/MAPK Pathway and Neuronal Growth

We have discovered that both the p75^{NTR} pathway and the MEK/MAPK pathway modulate neuronal growth. These findings allow us to provide assays for drugs which affect neuronal growth by modulation of these two signaling pathways. Such assays may measure various aspects of signaling components that include changes in: (a) phosphorylation status; (b) kinase activity; (c) neuronal growth; and (d) mRNA or polypeptide levels of components of either the p75^{NTR} or MEK/MAPK pathway. These measurements, which can be made in vitro or in vivo, form the basis of assays which identify compounds that modulate neuronal growth. Such identified compounds may have therapeutic value, for example, in the treatment of neurodegenerative disease and neurological trauma.

Secondary Screens for Compounds that Modulate Neuronal Growth.

After test compounds that appear to have neuronal growth-modulating activity are identified, it may be necessary or desirable to subject these compounds to further testing. The invention provides such secondary confirmatory assays. For example, a compound that appears to increase neuronal growth in early testing is subject to additional assays to confirm that the levels of other cell markers of neuronal growth are reproducibly influenced by the compound. At late stages, testing is performed in vivo to confirm that the compounds initially identified to affect neuronal growth in cultured neurons has the predicted effect on in vivo neurons. In the first round of in vivo testing, neuronal growth is conducted in animals using well-known methods and then the compound is administered by one of the means described in the “Therapy” section immediately below. Results may be compared to transgenic mice lacking BDNF and/or p75^{NTR}, or those in which MEK/MAPK signaling has been inhibited. Neurons or neural tissue are isolated within hours to days following the insult, and are subjected to assays as described in the examples below. Such assays are well known to those skilled in the art. Examples of such assays include, but are not limited to: immunolabeling, measuring neurotransmitter levels, ceramide levels, or phosphorylation levels, or monitoring a change in the shape, number, or otherwise growth-related quantifiable characteristic exhibited by the neuron.

In addition to SCG neurons or other sympathetic neurons, other types of primary neurons, isolated by standard techniques, such as cortical neurons, hippocampal neurons, and motor neurons, may also be used (see, e.g., Brewer et al., Nature 363:265-266, 1993; Henderson et al., Nature 363:266-267, 1993).

Cellular levels of other biochemical markers may be employed as an indication that neuronal growth is modulated by a test compound. Measurement of a polypeptide, mRNA, PCR product, and reporter gene activity of a reporter operatively linked to a protein promoter involved in either the p75^{NTR} pathway or the MEK/MAPK pathway are also part of the invention.

The assays described herein can also be used to test for compounds that inhibit neuronal growth and increase cell death and hence may have therapeutic value in the treatment of neuroproliferative disease.

Test Compounds

In general, novel drugs that promote neuronal growth are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available, for example, from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, antibodies (or fragments thereof) may also be generated using standard techniques known in the art and screened for their efficacy in promoting neuronal growth using the techniques described herein.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their therapeutic activities for neurodegenerative disorders should be employed whenever possible.

When a crude extract is found to increase neuronal growth, further fractionation of the positive lead extract is necessary to isolate chemical constituents responsible for the observed effect. Thus, the goal of the extraction, fractionation, and purification process is the careful characterization and identification of a chemical entity within the crude extract having neuronal growth promoting activities. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using a mammalian neuronal growth model.

Below are examples of assays that are readily adapted to high-throughput systems useful for evaluating the efficacy of a molecule or compound useful in promoting neuronal growth.

Therapy

Compounds identified using any of the methods disclosed herein may be administered to patients or experimental animals with a pharmaceutically-acceptable diluent, carrier, or excipient, in unit dosage form. Conventional pharmaceutical practice may be employed to provide suitable formulations or compositions to administer such compositions to patients or experimental animals. Although intravenous administration is preferred, any appropriate route of administration may be employed, for example, parenteral, subcutaneous, intramuscular, intracranial, intraorbital, ophthalmic, intraventricular, intracapsular, intraspinal, intracistemal, intraperitoneal, intranasal, aerosol, or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in Remington: The Science and Practice of Pharmacy, (19th ed.) ed. Gennaro A R., 1995, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for antagonists or agonists of the invention include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel. In addition, specific techniques may be employed for delivering molecules across the bloodbrain barrier (BBB) of the central nervous system (CNS). For example, shunts for direct infusion into the brain may be employed as well as osmotic or pharmacologic disruption of the BBB (see, e.g., Kroll et al., Neurosurgery 42:1083-1100, 1998).

The following examples are provided to illustrate the invention and should not be construed as limiting.

### EXAMPLE 1

### General Methods

Neuronal Cell Culture

Mass cultures of pure sympathetic neurons from the superior cervical ganglion (SCG) of postnatal day 1 Sprague Dawley rats (Charles River Breeding Laboratories, St. Constant, Quebec, Canada) were prepared as previously described (Ma et al., J. Cell Biol. 117:135-141, 1992). Neurons were plated at low density (approximately one ganglion/well) in Nunclon™ 4-well culture dishes (Gibco BRL, Burlington, Ontario, Canada), coated with either rat tail collagen or poly-D-lysine and laminin (both from Collaborative Biomedical Products, Bedford, Mass.). Culture medium was UltraCulture (BioWhittaker, Walkersville, Md.), supplemented with 3% rat serum (Harlan Bioproducts, Madison, Wis.), 2 mM glutamine, 100 U/ml penicillin, 100 μg/ml streptomycin (all from BioWhittaker), and for the first two days, 7 μM cytosine arabinoside (Sigma-Aldrich Canada Ltd., Oakville, Ontario, Canada).

CD-1 mouse sympathetic neurons were cultured by a modification of the method used to prepare rat neurons. Mouse cultures were essentially prepared the same way, but were dissociated in UltraCulture medium rather than Hanks' balanced saline solution. 3% fetal bovine serum (Gibco) was used instead of rat serum, and 3.5 μM cytosine arabinoside was added to the culture medium on day one post-plating.

NGF used in these experiments was purified from mouse salivary glands and as supplied by Cedarlane Laboratories Ltd. (Hornby, Ontario, Canada). The sources of recombinant human BDNF were PeproTech Inc. (Rocky Hill, N.J.), for the neuritogenesis assays, and Promega Corporation, (Madison, Wis.), for the rhBDNF neutralization experiments. The p75^{NTR} function-blocking antibody REX is directed against the extracellular domain of p75^{NTR}, and was used as an antiserum at a dilution of 1:100 (Weskamp and Reichardt, Neuron 6:649-663, 1991). Rabbit serum (Gibco) of the same concentration was used as the negative control for REX. Anti-Human BDNF pAb (Promega), was used at 10 μg/ml. As a negative control for anti-BDNF, nonimmune chicken IgY (Promega) was used at up to 40 μg/ml in BDNF neutralization experiments, and 10 μg/ml in neuritogenesis experiments.

BDNF Neutralization

To test the capacity of anti-BDNF to neutralize BDNF, TrkB-expressing NIH-3T3 cells were cultured in Dulbecco's modified Eagle medium. Briefly, cells were washed twice and incubated for one hour at 37° C. in buffer, followed by a 30 minute wash at 37° C. in a phosphate-free buffer. Treatment consisted of incubating cells for five minutes with either 50 ng/ml BDNF (Promega) or with BDNF preabsorbed for four hours at 4° C. with increasing concentrations of the BDNF antibody (5-40 μg/ml). In addition, TrkB-3T3 cells were treated with medium only, or medium plus 40 μg/ml nonimmune chicken IgY. Following these treatments, cells were lysed, immunoprecipitated with anti-pan Trk (Hempstead et al., Neuron 9:883-896, 1992), and the immunoprecipitates analyzed for TrkB activation by Western blot analysis with phosphotyrosine antibody 4G10 (Upstate Biotechnology Inc., Lake Placid, N.Y.).

Neuronal Cell Survival Assays

NGF-dependent neurons were selected by culturing sympathetic neurons for five days in the presence of 50 ng/ml NGF. Neurons were washed three times for one hour each in neurotrophin-free media, and were then fed with media containing 10 ng/ml NGF alone, or various concentrations of NGF plus either 100 ng/ml BDNF, 10 μg/ml αBDNF, or a 1:100 dilution of antiserum containing the p75^{NTR} antibody, REX (Weskamp and Reichardt, Neuron 6: 649-663, 1991). Each condition was repeated in triplicate, and analysis of survival was performed 48 hours later by using nonradioactive cell proliferation (MTT) assays (Celltitre 96; Promega; Belliveau et al., J. Cell Biol. 136:375-388, 1997). Specifically, 50 μl of the MTT reagent was added to 500 μl media in each well, and incubated for two hours at 37° C. After aspiration of the MTT containing media, 100 μl of a 0.065N HCl/isopropanol mixture was added to each well to lyse the cells, and colorimetric analysis was performed using an ELISA reader. For rat sympathetic neurons, 10 ng/ml NGF represents 100% survival, therefore all other values were considered to be relative to 10 ng/ml NGF. For mouse neurons, 7.5 ng/ml NGF represents 100% survival, and was thus considered to be the 100% survival threshold for neuritogenesis experiments.

Analysis of Transgenic Animals

Mice heterozygous for a targeted mutation in the BDNF gene or homozygous for a targeted mutation in the p75^{NTR} gene were obtained from Jackson Laboratories (Bar Harbor, Me.). The BDNF^{-/-} mice were maintained in a C129/BalbC background. The p75^{NTR-/-} mice were originally generated in a C129 background, and were backcrossed into a C129 background before purchase from Jackson Laboratories and then maintained as homozygotes. Progeny from BDNF heterozygote crosses were screened for the mutant allele(s) using PCR.

Quantification of Neuronal Growth

Regulation of neuronal growth was analyzed by an in vitro neuritogenesis assay. Postnatal day one rat sympathetic neurons were cultured in 50 ng/ml NGF for two to three days in order to upregulate p75^{NTR}, whose increased expression in response to NGF occurs independently of neuronal survival. Following a one hour wash in neurotrophin-free medium, cultures were maintained for an additional two days in 10 ng/ml NGF plus or minus 100 ng/ml BDNF. Fields in sister cultures containing a similar number of neuronal cell bodies were then photographed, and neuritic process density was determined as described below. Since murine sympathetic neurons are more sensitive to NGF, requiring less to maintain 100% survival in vitro, experiments using mouse neurons were performed with a background of 7.5 ng/ml NGF in the medium instead of 10 ng/ml NGF, and were stained with Coomassie Blue for five minutes before being photographed.

Quantitative analysis was performed by using common statistics applied to random sets of lines and determining the number of intersection points per unit area. The network of neuritic processes, when viewed microscopically, appears as a random set of lines in a plane. Low density fields were photographed, and the number of visible intersections and bifurcations of cell processes per unit area was considered to be a quantitative measure of process density. Since the number of neurites is a direct function of the number of neurons, however, the number of intersections and bifurcations counted per unit area was normalized to the number of cell bodies in that same area of interest. For each experimental condition, 6-8 sampling windows (10 mm²) were photographed, and the Student's t test was used to determine the statistical significance of density differences between experimental groups. Results are expressed as the mean density plus or minus the standard error of the mean.

Another assay to assess neuronal growth is based upon the measurement of tubulin, the major protein component of neurites and axons. In one assay, neurons are lysed and quantitative dot blots are performed. Using this approach, we have demonstrated that there is a correlation between the amount of tubulin and the amount of growth, as measured using morphological criteria. ELISA, using the same tubulin antibody, may also be used.

A further assay to measure neuronal growth is performed as follows. Axons of cultured sympathetic neurons are immunostained with anti-tubulin antibody, and the percentage of surface area that is covered by tubulin-immunoreactivity is then measured. In a typical protocol, sympathetic neurons are grown for two days in NGF at similar densities on collagen-coated 48- or 96-well plates. They are then switched into 10 ng/ml NGF or 10 ng/ml NGF plus 100 ng/ml BDNF. Two days later, they are immunostained for tubulin and counterstained with Hoechst. The percentage area covered by tubulin and the number of Hoechst-positive nuclei are enumerated using an image analysis machine. Such an analysis provides statistically-significant data collection on the amount of neurite growth per neuron. For high-throughput screens, a plate reader may be employed to “read” the fluorescence from the two fluorochromes.

For illustrative purposes, two specific examples are provided below. These are not meant to be limiting.

High-Throughput Screening of Neuronal Growth

Assays for neuronal growth (e.g., neuritic growth, axonal growth, or dendritic growth) may, for example, be performed as follows. Ninety-six-well plates are coated for at least one hour with rat tail collagen (prepared in a dilution of 1% glacial acetic acid). After one hour, excess collagen at aspirated and allow to dry completely. Neurons (e.g., SCG neurons, or any other primary neuron or neuron derived from a cell line or stem cell) are placed into culture. At the desired endpoint of the growth experiment, neurons are washed briefly with PBS and then fixed for 10 minutes with 100 μl 4% formaldehyde (freshly prepared, BDH). Excess fixative is removed from cells by 3×5 min washed with 200 μl PBS. PBS is aspirated and replaced with 100 μl of staining solution (e.g., 10% acetic acid, 40% methanol, 0.25% (w/v) Coomassie Brilliant Blue R-250; “Coomassie Blue”) for 15 minutes. If Coomassie Blue is used, unbound stain can be removed by, for example, 3×10 min washed with 200 μl destaining solution (10% acetic acid, 40% methanol). Remaining wash solution is aspirated and 100 μl of solubilization solution is added (10% HCl in isopropanol). The plates can be gently agitated for five minutes to ensure complete solubilization. The concentration of bound dye (which is proportional to neuronal growth) is then measured on a absorbance spectrophotometer at the appropriate wavelength (e.g., 595 nm for Coomassie Blue).

Measurement of Dendritic Growth

Dendritic growth can be measured as follows. Ninty-six-well plates are coated for at least 24 hours with rat tail collagen (prepared in a dilution of 1% glacial acetic acid). After one hour, excess collagen is aspirated and allow to dry completely. SCG neurons are dissected, triturated and plated. Cells may also be plated onto 8-well chamber slides. Cells are fed after about three days and, after about five days, are switched to either 50 mM KCl or 10 ng/ml NGF+50 mM KCl for about three additional days. Neurons are washed briefly with PBS and then fixed for 10 minutes with 4% formaldehyde in PBS (freshly prepared). Excess fixative is removed by 3×5 min washes with PBS. Cells are stained for one hour at room temperature with the following antibodies with 3×5 min PBS washes after each incubation: primary antibody—mouse monoclonal IgG1 anti high molecular weight MAP2 (clone AP20, from Sigma), secondary antibody—donkey anti mouse CY3, minimum cross to mouse and rabbit (Jackson Immunochemicals). Cell are mounted and at least three fields of view are captured using an upright fluorescence microscope using 530 excitation/580 emission filters. For each individual neuron in a given field (minimum 250 neurons total), the distance of MAP2 positive staining from the perimeter of the cell body is measured. These measurements are performed and tabulated using NIH image. The tabulated data are then expressed as a population histogram, binning all the measured lengths into 2 μm bins. This analysis gives an overall measure of the extent of dendritic differentiation in the population.

The growth assay utilizes the detection of protein or mRNA as a measure of the amount of neuronal growth. Thus any reagent that is suitable for the detection of protein will also be suitable for use in the present methods. Reagents suitable for a high-throughput growth assay include, for example, Bradford/Lowry reagent, BCA reagent, Commassie Brilliant Blue G-250 and R-250, colloidal Coomassie Blue, and sulforhodamine 101 (which is fluorescent). Other dyes are described in chapter 9 of the Molecular Probes Handbook (http://www.molecularprobes.com). Antigens suitable for labeling neurites include, for example, alpha or beta tubulin, GAP43, neurofilament (light, medium and heavy forms), MAP1B, MAP4, and juvenile MAP2 (e.g., MAP2C, MAP2D). Antigens that are preferentially found in dendrites include, for example, adult MAP2 (MAP2A, MAP2B), adult MAP2 mRNA, dendrin, dendrin mRNA, polyribosomal stain (Nissel stain), CaMKII mRNA, microtubules of mixed polarity (visualized by tannic acid fixation under electron microscopy), mGluR1 a receptor, mGluR2 receptor, and the 5-HT1a receptor. Note that several mRNAs (such as those listed above) are specifically located in dendrites, and these thus serve as markers of dendritic growth. Antigens that are preferentially found in axons include, for example, phosphorlated MAP1B, unipolar microtubules, 5-HT1b receptor, and phosphorylated neurofilament (heavy form only).

Those in the art will recognize that any neuronal culture method is compatile with the screening methods described herein, including dissociated neurons, organotypic neuronal cultures, and reaggregate neuronal cultures. As described above, any neuron or neuronal cell line that can be cultured and that forms neurites (e.g., axons, dendrites, or both) is suitable for use in the methods described herein.

Immunohistochemistry

For analysis of sympathetic innervation density in pineal glands of transgenic mice and control litter mates, animals were anaesthetized with sodium pentobarbital (35 mg/kg) or isofluorane, and killed by decapitation. Pineal glands were removed immediately and processed for immunohistochemistry as follows: tissue was immersion-fixed overnight (at 4° C.) in 4% formaldehyde in phosphate buffer (PB, pH 7.4), and subsequently cryoprotected in graded sucrose solutions. Twelve micron sections were cut on a cryostat and thaw-mounted onto chromalum-subbed slides. Sections were post-fixed in 4% formaldehyde in PB for 10 minutes at room temperature, and then washed for 10 minute in phosphate buffered saline (PBS, pH 7.4). Following non-specific blocking with 4% goat serum and 4% rat serum (both from Jackson ImmunoResearch Laboratories, West Grove, Pa.) plus 0.2% Triton X-100 in PBS (pH 7.4), pineal sections were incubated overnight at 4° C. with a commercially available polyclonal antibody directed against tyrosine hydroxylase (1:400, Chemicon International, Temecula, Calif.) in blocking solution. Slides were then washed three times (10 minutes each) in PBS, and incubated for two hours in blocking solution containing a CY3-conjugated secondary antibody (goat anti-rabbit IgG, 1:2000, Jackson ImmunoResearch). Following 3×10 minute washes in PBS, slides were coverslipped using Sigma Mounting Medium (Sigma Diagnostics, St. Louis, Mo.), and innervation density viewed by epifluorescence microscopy. For analysis of p75^{NTR} levels in sympathetic terminals in the pineal gland, pineal glands from P6 or adult Sprague Dawley rats were processed and visualized as above, but the primary antibody used was MC192 (Chandler et al., J. Biol. Chem. 259:6882-6889, 1984), which is directed against the extracellular domain of p75^{NTR}. The secondary antibody was a CY3-conjugated goat anti-mouse IgG used at a concentration of 1:2000 (Jackson Immunoresearch).

Immunoblot Analysis

For biochemistry, pineal glands or cortices were homogenized in Tris buffered saline (TBS) containing 137 mM NaCl, 20 mM Tris (pH 8.0), 1% v/v NP-40, 0.1% SDS, 10% glycerol and the protease inhibitors phenylmethyl sulfonyl fluoride (PMSF, 1 mM), aprotinin (10 μg/ml), leupeptin (0.2 μg/ml), and sodium vanadate (1.5 mM). The tissue was rocked for 10 minutes at 4° C., and following a 10 minute centrifugation at 4° C., the supernatant was collected and lysates normalized for protein concentration using a BCA Protein Assay kit (Pierce, Rockford, Ill.). p75^{NTR} protein was immunoprecipitated with of anti-recombinant human p75^{NTR} (Promega) per 500 μl lysis buffer. The immunoprecipitates were collected with protein A-Sepharose™ beads (Pharmacia Biotechnology Inc., Piscataway, N.J.) for 1.5 hours at 4° C., followed by centrifugation. For immunoblot analysis, equal amounts of protein were boiled in sample buffer for five minutes, and separated by 7.5% or 15% SDS-PAGE (7.5% for tyrosine hydroxylase and p75^{NTR}, and 15% for BDNF). After electrophoresis, proteins were transferred onto 0.2 μm nitrocellulose membranes for 1.5 hours at 0.6 amps, and washed three times (10 minutes each) with PBS. Following a 1.5 hour block in blotto (3% nonfat milk in PBST) at room temperature, membranes were incubated overnight at 4° C. in blocking solution containing either anti-BDNF (1:3000; Santa Cruz Biotechnology Inc., Santa Cruz, Calif.), anti-tyrosine hydroxylase (1:5000; Chemicon), or anti-p75^{NTR} (1:10,000; Promega). The membranes were washed four times with TBST (10 minutes each wash), and then incubated with secondary antibody (1:10,000 goat anti-rabbit HRP, Boehringer Mannheim, Laval, Quebec, Canada) for 1.5 hours. After three washes in PBST, detection was carried out using enhanced chemiluminescence (Amersham Canada Ltd., Oakville, Ontario, Canada) and XAR X-ray film (Eastman Kodak Co., Rochester, N.Y.).

### EXAMPLE 2

### BDNF-mediated Activation of p75^{NTR} Inhibits NGF-induced Growth of Cultured Sympathetic Neurons

BDNF-mediated activation of p75^{NTR} antagonizes TrkA-mediated sympathetic neuronal survival when NGF levels are suboptimal, but has no effect on survival at higher levels of NGF. To determine whether p75^{NTR} activation also antagonized other TrkA-mediated biological responses, we focused on sympathetic neuronal growth. Specifically, we cultured sympathetic neurons in 10 ng/ml NGF and then activated p75^{NTR} using BDNF. For rat sympathetic neurons, 10 ng/ml NGF mediates 100% sympathetic neuron survival but elicits limited morphological growth and TrkA activation relative to higher concentrations of NGF, while 100 ng/ml BDNF is sufficient to activate p75^{NTR} in apoptosis experiments, but does not bind to the two Trk receptors present on sympathetic neurons, TrkA and TrkC.

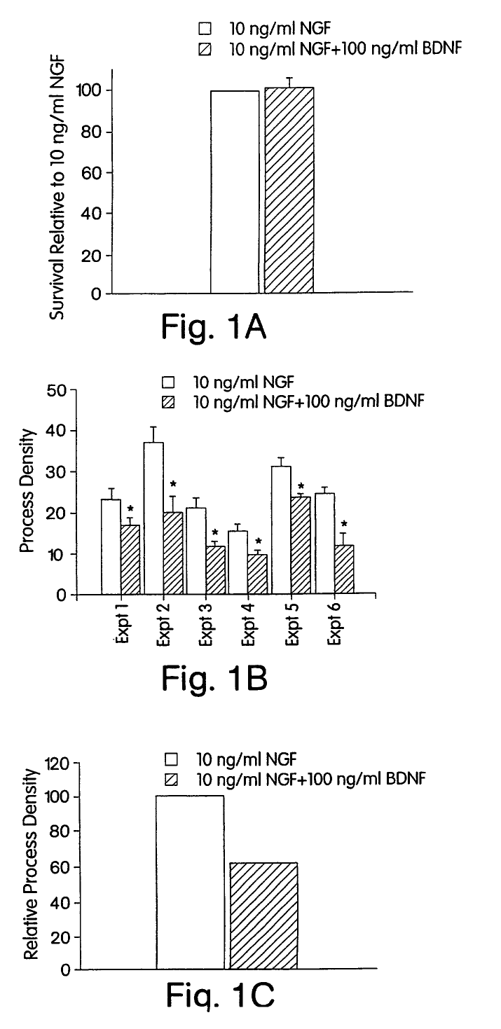

Initially, we observed that the addition of 100 ng/ml BDNF in the presence of 10 ng/ml NGF had no negative effects on sympathetic neuron survival (FIG. 1A). Specifically, sympathetic neurons were cultured for five days in 50 ng/ml NGF, were free of neurotrophin, and then switched into 10 ng/ml NGF plus or minus 100 ng/ml BDNF. Two days later, neuronal survival was measured using MTT assays, which measure mitochondrial function. As previously shown, the addition of 100 ng/ml BDNF had no effect on sympathetic neuron survival in 10 ng/ml NGF (FIG. 1A).

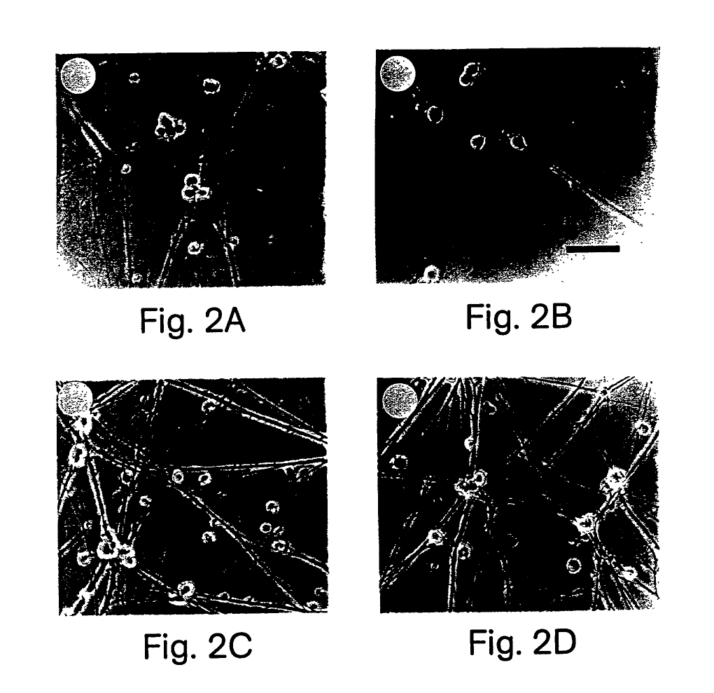

We next determined whether p75^{NTR} activation affected neuronal growth by measuring the level of neurite extension that occurs in response to 10 ng/ml NGF with or without BDNF. For these experiments, neurons were cultured for two to three days in 50 ng/ml NGF, were switched to 10 ng/ml NGF plus or minus 100 ng/ml BDNF, and the density of neuritic processes was then determined two days later (FIGS. 1B, 1C). Results from six separate experiments indicated that p75^{NTR} activation reduced the process network density from 22 to 52%, for an average decrease of 40% (FIGS. 1B, 1C, 2A, 2B).

### EXAMPLE 3

### Autocrine BDNF, Acting through p75^{NTR} Decreases Growth of Cultured Sympathetic Neurons

The foregoing data suggested that exogenous BDNF was able to activate p75^{NTR} and negatively-influence TrkA-mediated neuritogenesis. Since sympathetic neurons themselves synthesize BDNF that can be detected in conditioned medium obtained from cultured SCG neurons, we hypothesized that autocrine BDNF might play a role in negatively regulating levels of sympathetic neuron growth through an autocrine loop. To test this hypothesis, cultures were maintained for two to three days in 50 ng/ml NGF, and then cultured for two more days in 10 ng/ml NGF, plus or minus a function-blocking anti-BDNF antibody. As a control, we used a nonimmune control chicken IgY.

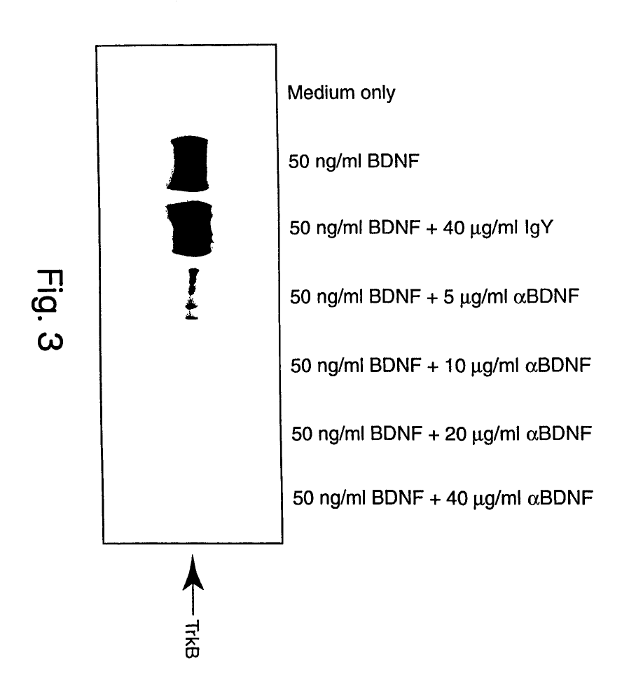

To ensure that the anti-BDNF was capable of neutralizing BDNF, we incubated TrkB-expressing NIH-3T3 cells for five minutes with 50 ng/ml BDNF plus or minus 5-40 μg/ml of anti-BDNF. TrkB protein was then immunoprecipitated using anti-panTrk, and the immunoprecipitates analyzed by immunoblot analysis with anti-phosphotyrosine. As controls, cells were incubated either with culture medium, or medium with BDNF plus 40 μg/ml nonimmune IgY. This analysis revealed that BDNF caused a robust increase in tyrosine phosphorylation of TrkB (FIG. 3, lanes 1, 2), and anti-BDNF inhibited BDNF-stimulated TrkB phosphorylation at concentrations of 10 μg/ml or higher (FIG. 3, lanes 4-7). In contrast, the control IgY had no effect on BDNF-mediated TrkB activation (FIG. 3, lanes 2, 3).

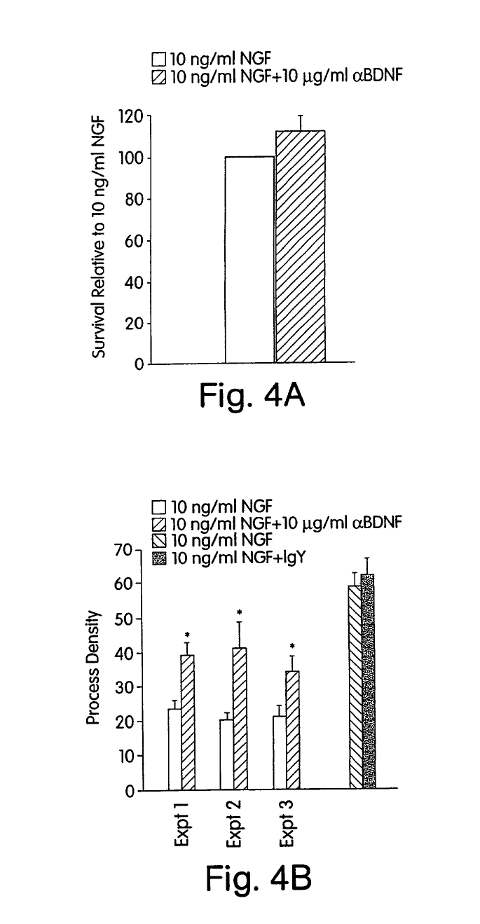

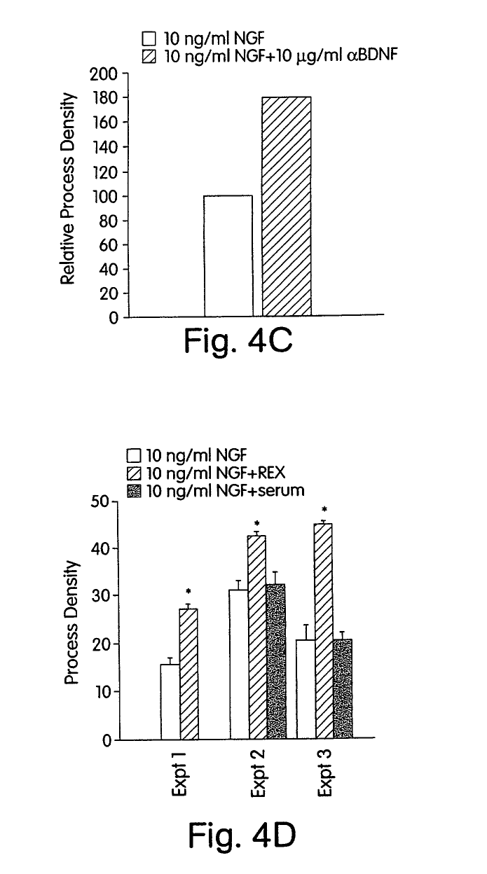

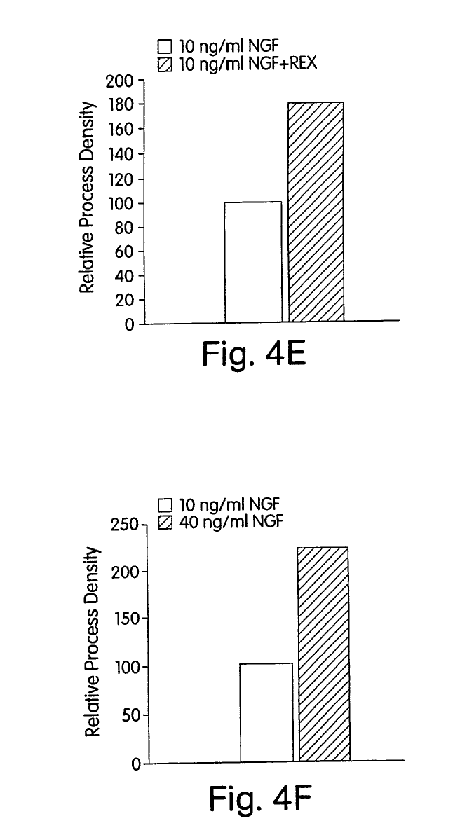

We then used this function blocking anti-BDNF to test the role of autocrine BDNF in sympathetic neuron growth. Initially, we determined whether anti-BDNF had any effect on sympathetic neuron survival in 10 ng/ml NGF. Neurons were cultured for five days in 50 ng/ml NGF, and then were switched to 10 ng/ml NGF with or without 10 μg/ml anti-BDNF. Measurement of neuronal survival using MTT assays two days later revealed that anti-BDNF had no effect on sympathetic neuron survival under these conditions (FIG. 4A). We then determined whether anti-BDNF affected neuronal growth under these same conditions; neurons were grown in 50 ng/ml NGF for two to three days, and then switched into 10 ng/ml NGF plus or minus 10 μg/ml anti-BDNF. Measurement of neurite process density revealed that cultures exposed to anti-BDNF exhibited an average increase in neuritogenesis of 80%, relative to 10 ng/ml NGF alone (FIGS. 2A, 2C, 4B, 4C). Nonimmune IgY had no effect on NGF-mediated growth (FIG. 4B), demonstrating the specificity of the effect. Thus, autocrine BDNF inhibits TrkA-mediated neuritogenesis in vitro.

Since we observed autocrine BDNF was mediating these effects via p75^{NTR}, we predicted a similar increase in TrkA-mediated neuritogenesis if we blocked p75^{NTR}. To test this prediction, we performed neuritogenesis experiments using the function-blocking p75^{NTR} antibody, REX. As before, cultures were initially grown for two days in 50 ng/ml NGF and were then incubated for two additional days with 10 ng/ml NGF with or without REX (FIGS. 2A, 2D, 4E, 4F). As a control, sister cultures were incubated with rabbit serum at the same volume as the REX antiserum. These experiments revealed that, when p75^{NTR} was blocked by REX, neuronal growth was enhanced almost two-fold relative to NGF alone (FIGS. 2A, 2D, 4E, 4F), an effect that was not observed with rabbit nonimmune serum (FIG. 4D), and that is similar to the response elicited by anti-BDNF (FIGS. 4B, 4C). The increased neuronal growth observed with both REX and anti-BDNF is similar to the 2 to 2.5-fold increase that occurs when NGF is increased from 10 to 40 ng/ml NGF (FIG. 4G), a treatment that causes increased TrkA activation, supporting the idea that a BDNF:p75^{NTR} autocrine loop antagonizes TrkA-mediated sympathetic neuron growth.

### EXAMPLE 4

### p75^{NTR-/-} Sympathetic Neurons Show Enhanced Neuronal Growth In Response to NGF and Do Not Respond to Exogenous BDNF

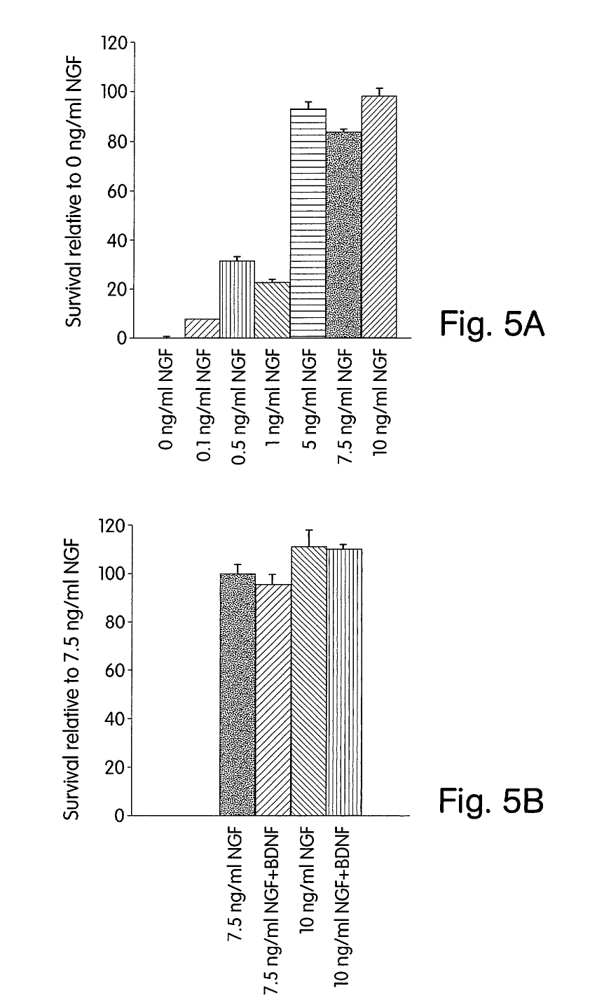

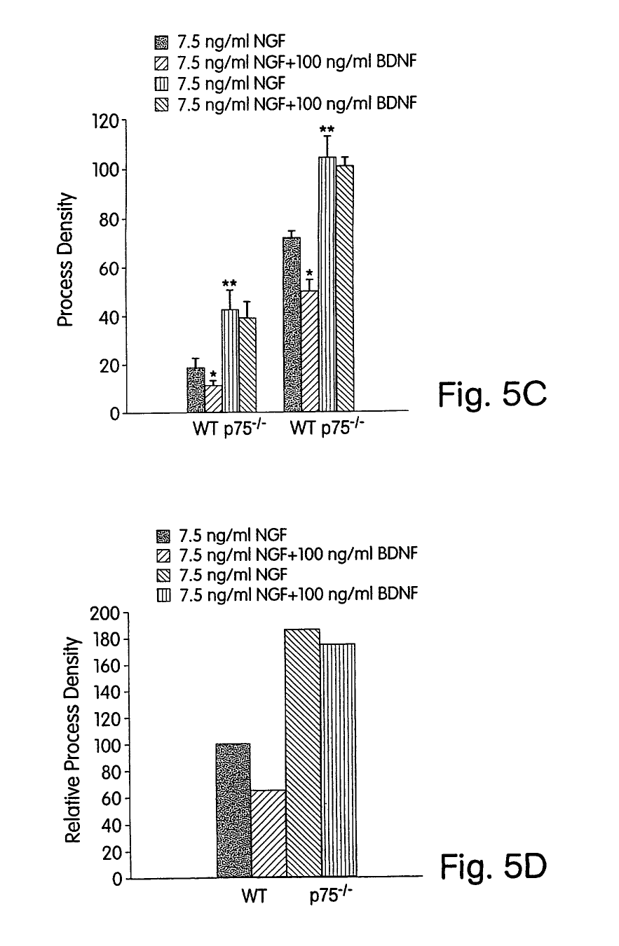

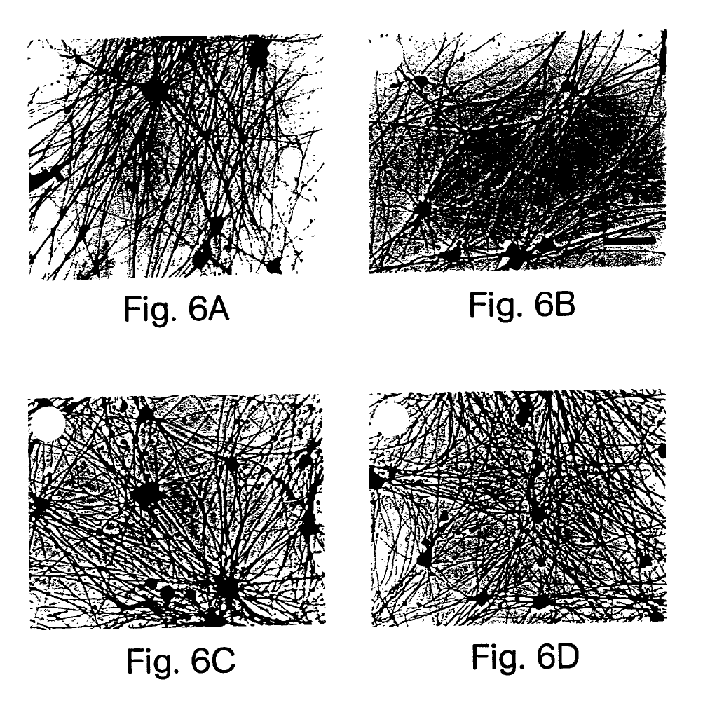

Our data strongly indicate that BDNF acts through p75^{NTR} to antagonize Trk-mediated enhanced neuronal growth. To formally demonstrate the necessity of p75^{NTR} for BDNF's effects, we cultured neurons from both p75^{NTR-/-} and wild type control mice and repeated the neuronal growth assays. Since mouse sympathetic neurons are more sensitive to NGF than rat sympathetic neurons, we initially performed survival assays to determine an appropriate NGF concentration. Specifically, mouse sympathetic neurons were maintained for five days in 50 ng/ml NGF, were switched to concentrations of NGF ranging from 0.1 to 10 ng/ml NGF for three days, and survival was then measured using MTT assays. These experiments revealed that 5, 7.5, and 10 ng/ml NGF were all able to mediate maximal mouse sympathetic neuron survival (FIG. 5A). To ensure that BDNF had no apoptotic effect under these survival conditions, we performed similar experiments with 7.5 or 10 ng/ml NGF plus or minus 100 ng/ml BDNF. MTT assays revealed that BDNF did not affect mouse sympathetic neuron survival in the presence of optimal concentration of NGF (FIG. 5B). On the basis of these data, we selected 7.5 ng/ml NGF for our experiments, a concentration that was optimal for survival and where BDNF had no significant effect on survival. We then examined sympathetic neurons from p75^{NTR-/-} mice to determine first, whether the lack of p75^{NTR} imparted to p75^{NTR-/-} neurons an intrinsic ability to extend more neuritic processes, and second, whether BDNF was acting through p75^{NTR} to inhibit TrkA-mediated growth. To perform these experiments, sympathetic neurons were cultured from p75^{NTR-/-} versus control mice on the same day, were maintained for three days in 50 ng/ml NGF, and were subsequently switched to 7.5 ng/ml NGF, plus or minus 100 ng/ml BDNF. Measurement of neuritic process density revealed that p75^{NTR-/-} neurons exhibited an almost two-fold increase in neuronal growth relative to their wild type counterparts (FIGS. 5C, 5D, 6A, 6C), a result similar to that observed with the REX and anti-BDNF antibodies in experiments using rat sympathetic neuron cultures (FIGS. 4B-4E). Moreover, while BDNF decreased the degree of neuronal growth in wild type mouse cultures by an average of 35%, exogenous BDNF had no effect on growth of p75^{NTR-/-} neurons (FIGS. 5B, 5C, 6C, 6D). Thus p75^{NTR} is required for BDNF to inhibit NGF-mediated sympathetic neuronal growth, and NGF is more effective at eliciting growth in the absence of p75^{NTR}.

### EXAMPLE 5

### BDNF is Present in Sympathetic Target Organs and p75^{NTR} in Sympathetic Neuron Axons During the Period of Target Innervation

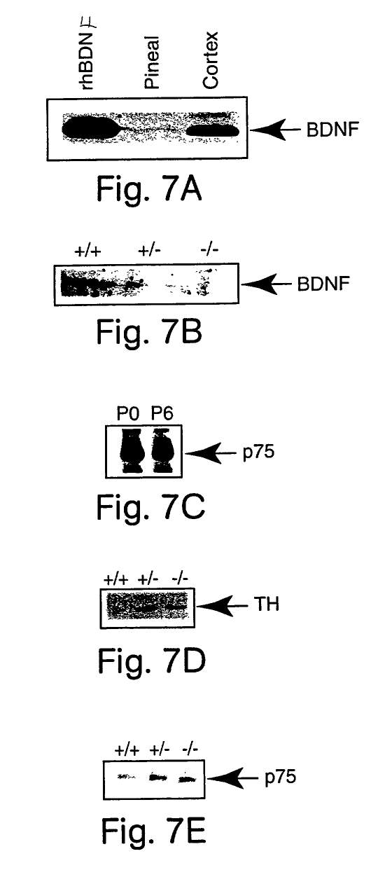

Our culture results indicate that BDNF would be present in sympathetic neuron targets during the developmental period of target competition. To test this experimentally, we focused on the pineal gland, a sympathetic target organ that (i) is bilaterally innervated by neurons from the SCG, (ii) does not receive any other peripheral innervation from sensory or motor neurons, and (iii) is innervated postnatally. Ingrowth of sympathetic fibers to the pineal gland begins during the first week of postnatal life, reaching adult levels after 3-4 weeks. Lysates of pineal glands from adult rats were separated by polyacrylamide gel electrophoresis, transferred to nitrocellulose, and probed for the presence of BDNF. This analysis revealed a BDNF-immunoreactive band in the pineal gland that is the same size as BDNF in the rat cortex, and human recombinant BDNF (FIG. 7A). To confirm the identity of this band, we analyzed the pineal gland from mice in which the BDNF gene was mutated by homologous recombination. Immunoblot analysis revealed that the BDNF-immunoreactive band was decreased in the pineal glands of P13 to P15 BDNF^{+/-} mice, and was completely lost in the pineal glands of BDNF^{-/-} mice (FIG. 7B). Thus, BDNF is present in the pineal gland at the time of sympathetic target competition.

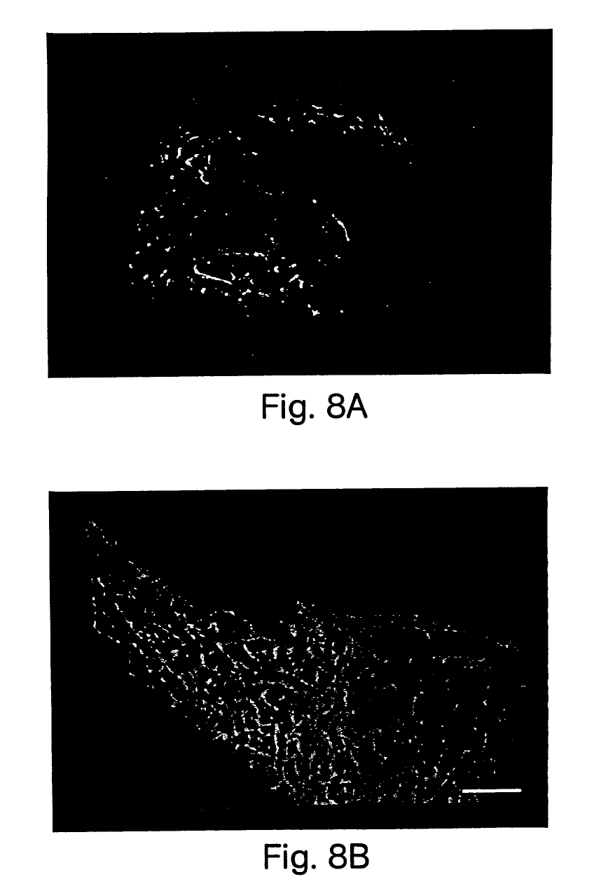

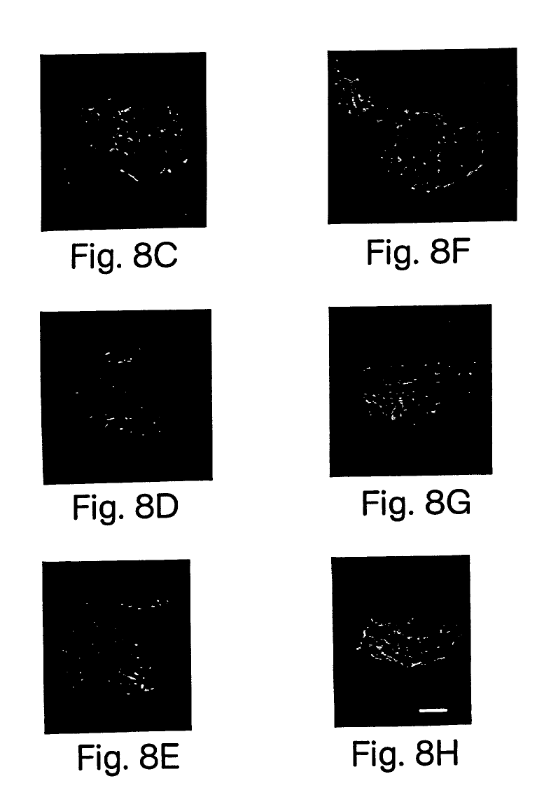

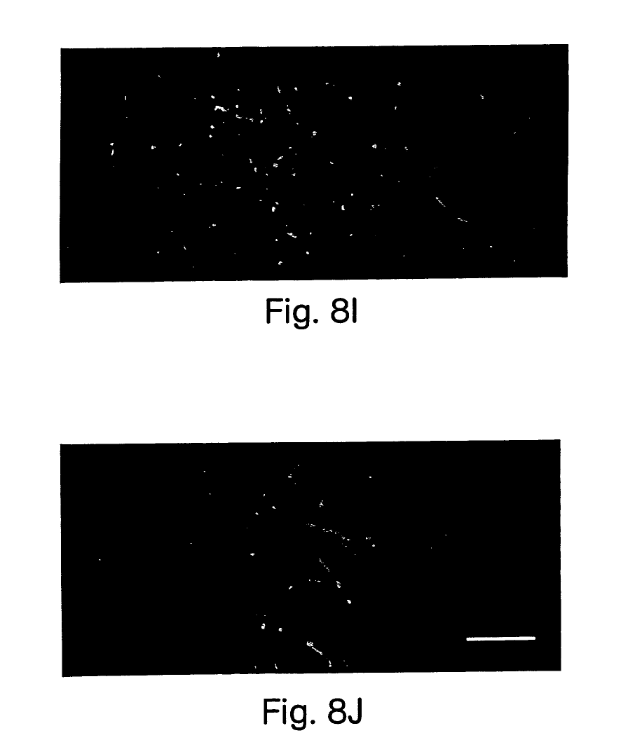

We next determined whether incoming sympathetic axons were positive for p75^{NTR} over this same time frame. Immunohistochemical analysis of the rat pineal gland using the anti-p75^{NTR} antibody MC192 revealed the presence of numerous p75^{NTR} positive fibers throughout the pineal gland at P6 (FIG. 8J), in a pattern similar to that previously observed for tyrosine hydroxylase-positive sympathetic fibers. This pattern was somewhat distinct from that observed in the adult pineal gland, in which p75^{NTR} immunostaining is more punctate in nature, reflecting the mature pattern of sympathetic fibers in the pineal gland (FIG. 8I), as observed for TH-positive sympathetic fibers. To confirm that this immunostaining corresponded to p75^{NTR}, we also performed immunoblot analysis, which demonstrated that p75^{NTR} was present in the pineal gland during the first few postnatal weeks (FIG. 7C). Thus, both BDNF and p75^{NTR} are present in the pineal gland at the time of sympathetic target competition.

### EXAMPLE 6

### The Pineal Gland is Hyperinnervated in BDNF^{-/-} Mice

Together, our in vivo and in vitro data predict that, in the absence of BDNF, sympathetic neuron target innervation should be increased. To test this experimentally, we examined the level of sympathetic innervation to the pineal gland of BDNF^{-/-} mice at P13 to P15. Initially, we analyzed the density of sympathetic fibers using immunohistochemistry for tyrosine hydroxylase, which is specific for sympathetic axons. This analysis revealed that the plexus of TH-positive fibers was much more dense in the pineal gland of BDNF^{-/-} mice than in the pineal gland of control litter mates (FIGS. 8A-8H). In particular, thick, TH-positive fibers were interspersed throughout the entire tissue in BDNF^{-/-} mice, whereas fibers in the pineal gland of control litter mates were less abundant and appeared qualitatively thinner.

To confirm this increase in TH-positive fibers, we also measured the level of TH in the pineal gland biochemically at P13 to P15. Western blot analysis revealed that TH levels were significantly increased in the pineal gland of BDNF^{-/-} animals relative to their control litter mates (FIG. 7D). TH levels were similarly increased in the pineal gland of BDNF^{+/-} litter mates (FIG. 7D), coincident with decreased BDNF (FIG. 7B), indicating that the increased sympathetic innervation was not due to any potential developmental delay in the BDNF^{-/-} mice. Moreover, p75^{NTR} levels were similar in the pineal glands regardless of the BDNF gene dosage (FIG. 7E), indicating that the differences in levels of innervation are not due to differences in levels of this receptor. Thus, when BDNF is either reduced or absent, both the density of sympathetic innervation and levels of TH are increased.

### EXAMPLE 7

### Genetic Perturbation of NGF-Mediated Neuronal Growth

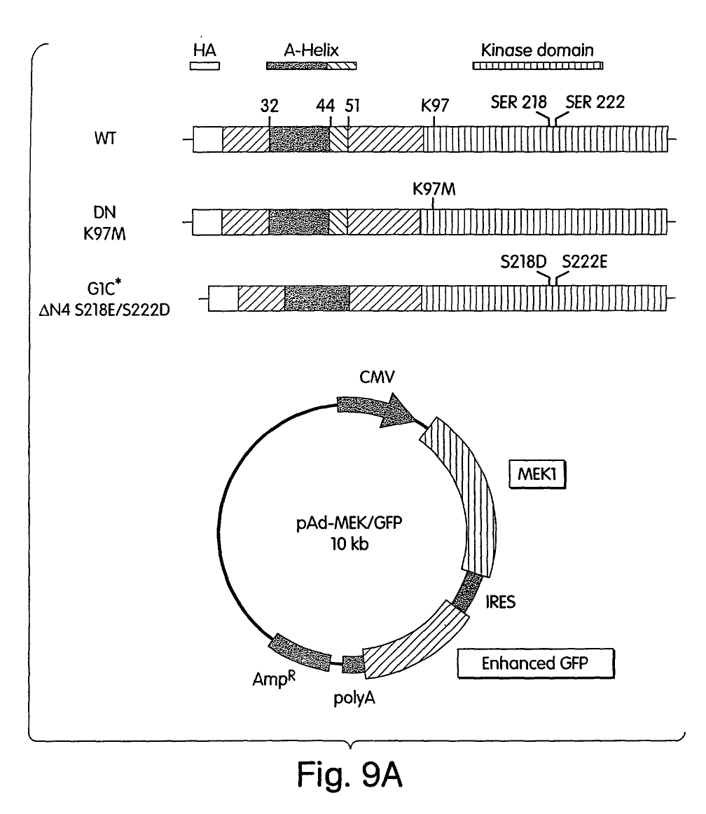

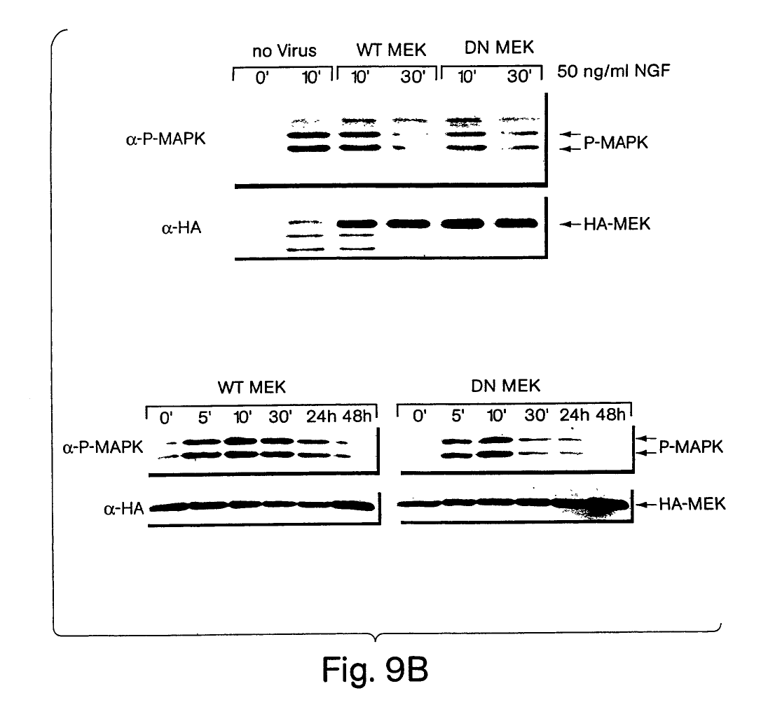

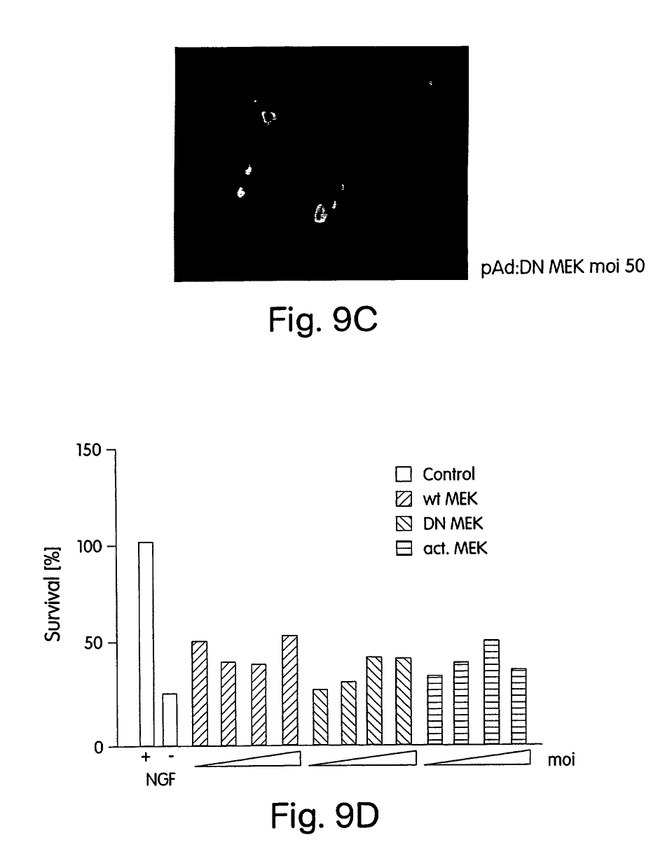

We have presently discovered that MEK/MAPK signaling modulates neuronal growth, without modulating cell survival. Accordingly, a compound that blocks MEK/MAPK signaling would block neuronal growth without affecting cell survival. One mechanism for blocking or inhibiting MEK/MAPK signaling is to use a dominant-negative MEK protein, such as DN MEK (FIG. 9A), in which lysine 97 has been replaced with a methionine (see, for example Holt et al., Mol. Cell Biol. 16:577-583, 1996). Using such an approach we demonstrated that MEK/MAPK signaling is required for neuronal growth both in vitro and in vivo.

We first inhibited MEK activity in vitro using a recombinant adenovirus expressing DN MEK. We also examined the role of MEK in vivo by generating transgenic mice which express DN MEK under the control of a neuron-specific and pan-neuronal Tα1 α tubulin promoter (Tα1: DN MEK).

Nucleic acid molecules encoding one of three HA-tagged MEK were cloned into an adenoviral vector which expresses green fluorescent protein (GFP) in a dicistronic fashion. WT MEK encoded wild-type MEK, DN MEK, in which lysine 97 was replaced with a methionine, encoded a dominant negative MEK, and ACT MEK, in which serine 218 and serine 222 were replaced by aspartic acid and glutamic acid, respectively, encoded an activated MEK (FIG. 9A).

### EXAMPLE 8

### DN MEK-Infected Cells Show Decreased MAPK Activation Following Treatment with NGF

Within 10 minutes of treatment with NGF, PC12 cells show marked increase in the phosphorylation of MAPK; prior infection of PC12 cells with an adenovirus encoding WT MEK did not diminish this phosphorylation event (FIG. 9B). In contrast, prior infection of PC12 cells with DN MEK resulted in a dramatic decrease in MAPK phosphorylation, as determined by western blotting with an antibody that specifically recognizes phosphorylated MAPK (P-MAPK), and not unphosphorylated MAPK (FIG. 9B). This decrease in P-MAPK is not due to an overall decrease in protein, as western blots of WT MEK and DN MEK-infected cells with anti-HA revealed the same amount of HA-tagged protein.

Neurons isolated from rat superior cervical ganglia were also infected with either WT MEK or DN MEK. As demonstrated for the PC12 cells, DN MEK decreased levels of MAPK phosphorylation (FIGS. 9B, 9C).

### EXAMPLE 9

### Survival of SCG Neurons in the Absence of NGF is not Enhanced Following Infection with MEK-Encoding Adenovirus

One explanation for the observed decrease in P-MAPK levels in SCG neurons was that expression of various MEK constructs altered cell survival. We performed a survival assay designed to detect survival-altering activity of MEK after NGF withdrawal. None of the constructs showed a significant alteration in cell survival (FIG. 9D). These data indicate that DN MEK reduced MAPK phosphorylation without altering cell survival.

### EXAMPLE 10

### DN MEK Decreases Neuronal Growth

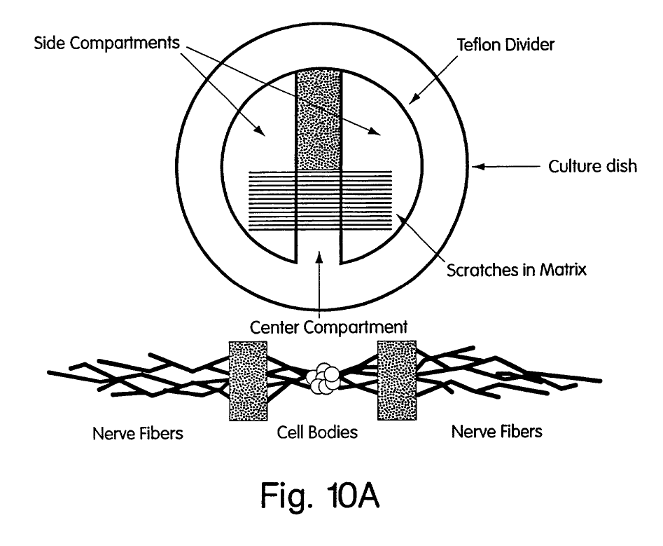

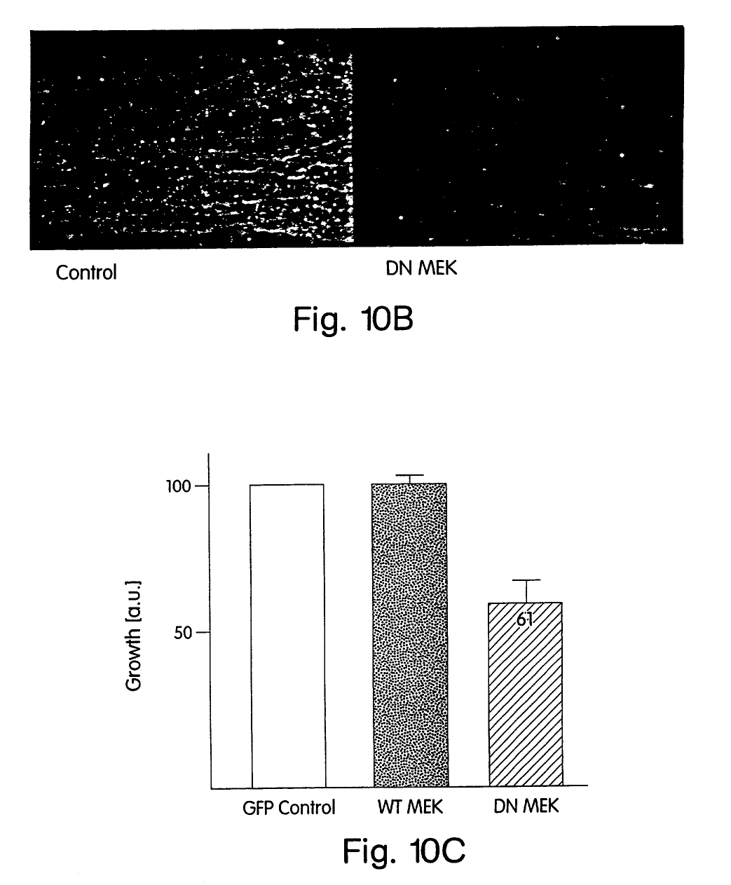

We have devised a method for quantitatively assaying neurite growth. In this assay, the cell bodies of neurons are plated in Campenot chamber, a compartmentalized dish that has scratches or grooves in the matrix. The compartments are physically separated from each other by, for example, a Teflon® divider. The cells are placed in the central compartment. The scratches allow neurites to extend from the neuronal cell bodies and into adjacent compartments (FIG. 10A).

Neurites were induced to grow into HGF supplemented side compartments, and then visualized by staining against tubulin. Adenoviral-mediated expression of DN MEK in SCG neurons decreased neuritogenesis by 39% (FIGS. 10B, 10C).

### EXAMPLE 11

### Generation and Phenotvpe of Transgenic Mice that Express DN MEK

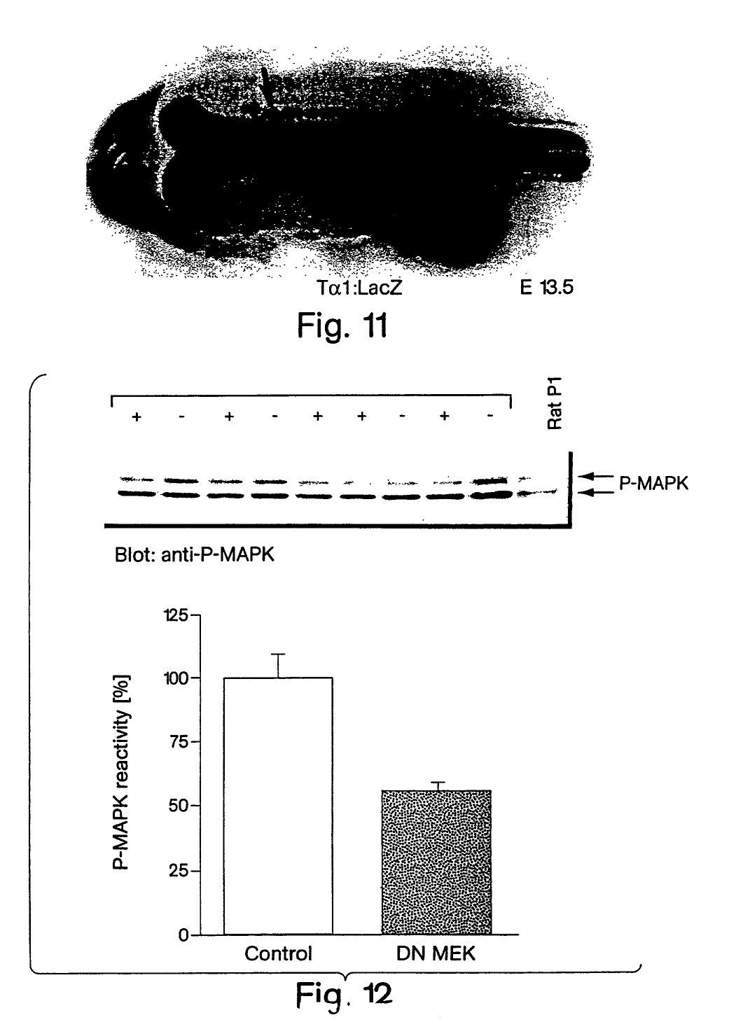

Using standard techniques, we generated five lines of transgenic mice that express DN MEK under the control of the Tα1 α tubulin promoter. FIG. 11 shows a beta-galactosidase-stained embryo at E13.5 of Tα1: lacZ reporter mouse, demonstrating that the Tα1 α tubulin promoter is targeted and limited to the central and peripheral nervous systems. Peak of expression was found to be at E16.5, with declining levels thereafter.

In order to determine whether DN MEK decreased P-MAPK levels, we analyzed proteins from tissue lysates of hippocampi dissected from transgenic P4 mice and nontransgenic controls. Densitometric quantification showed that transgenic expression of DN-MEK reduces P-MAPK reactivity in the hippocampus to approximately 50% of the wild-type value (FIG. 12).

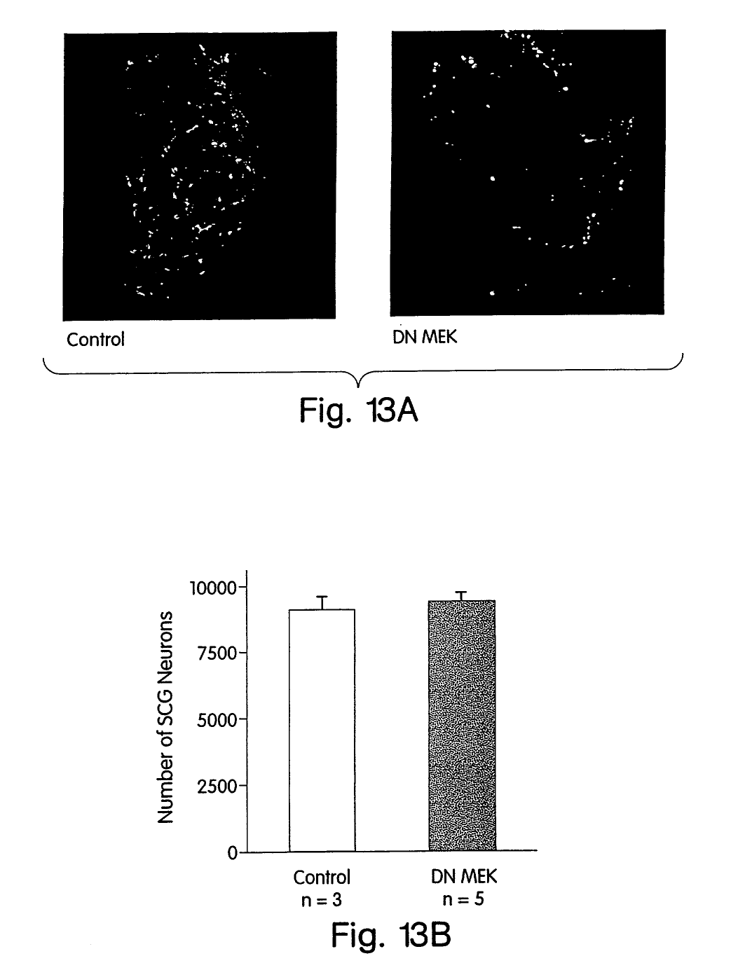

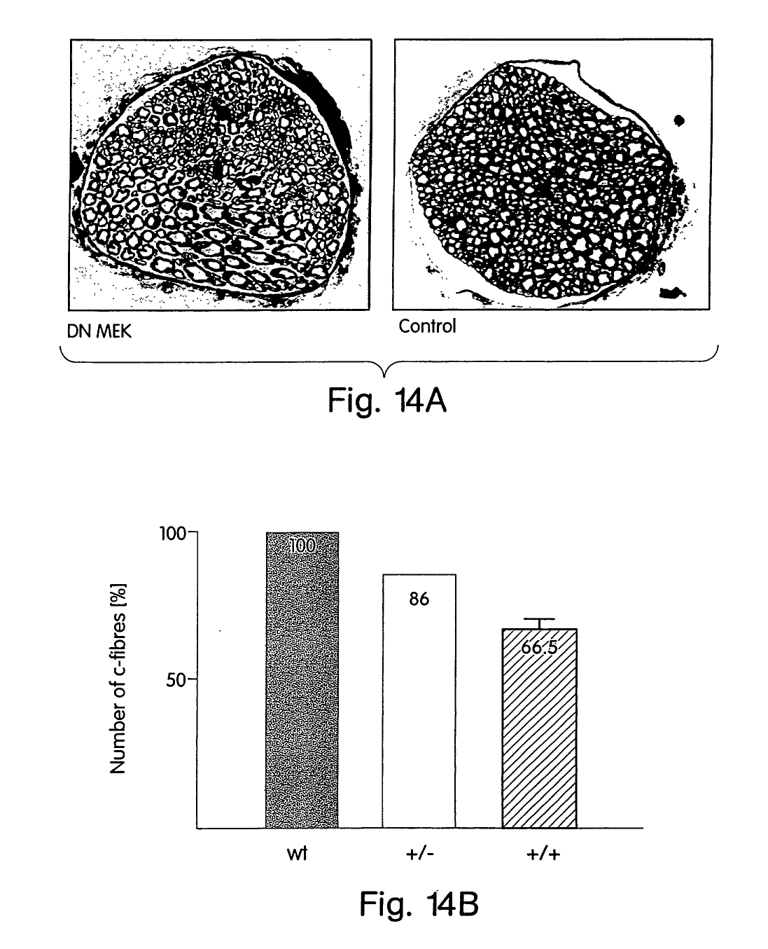

We examined the sympathetic innervation of pineal glands by immunohistochemistry against tyrosine hydroxylase (TH). Transgenic liter mates showed a significant loss of sympathetic innervation compared to non-transgenic controls (FIG. 13A). Computer-based imaging was used to quantify innervation. Using this method, we determined that sympathetic innervation of the pineal gland was reduced by approximately 75%. Loss of sympathetic innervation of the pineal gland is not due to cell death. By counting sympathetic neurons of the SCG, we found no difference between wild-type and transgenic litter mates. We conclude that loss of pineal gland innervation in DN MEK transgenic animals is not due to increased cell death of SCG neurons (FIG. 13B).

Tα1 :DN MEK mice also have a spatial loss of unmyelinated C-fibers in the medial branch of the dorsal cutaneous artery. Counting of axons revealed a loss of approximately 35% of unmyelinated axons (C-fibers) (FIGS. 14A, 14B), while the number of myelinated axons remain constant.

### EXAMPLE 12

### Pharmacologic Inhibition of MEK Activity Blocks Trk-Mediated Neuronal Growth in Primary Neurons

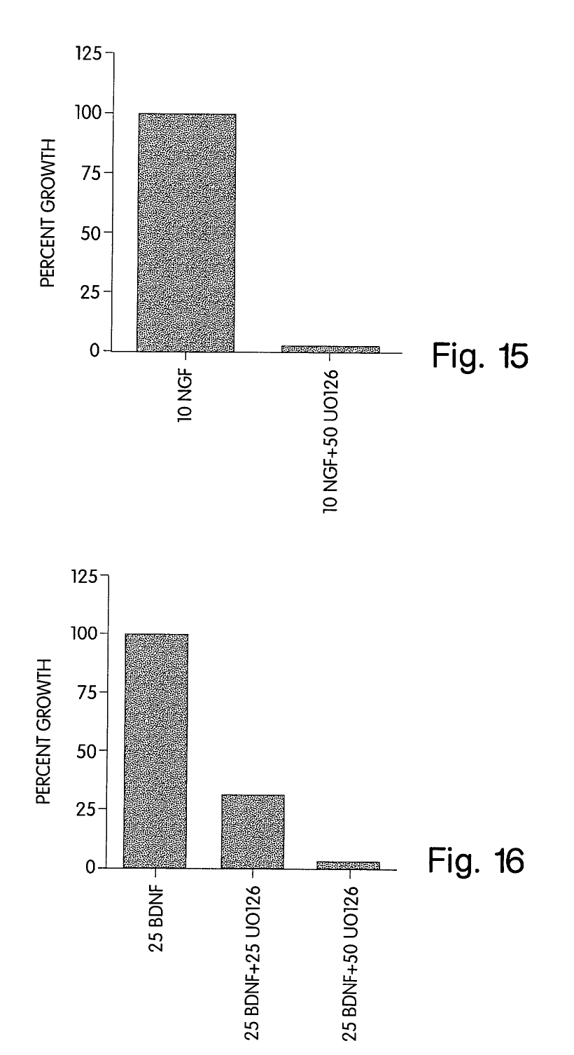

We cultured postnatal sympathetic neurons in Campenot chambers. Cells exhibit robust neuronal growth into a side compartment containing 10 ng/ml NGF. Addition of 50 μM U0126 in conjunction with 10 ng/ml NGF abrogates this growth while having no effects on cell survival (FIG. 15).

MEK activity is also required to transduce TrkB-mediated neuronal growth. BDNF does not induce neuronal growth of sympathetic neurons because the neurons lack TrkB receptors. We transduced postnatal sympathetic neurons with an adenovirus encoding TrkB. In Campenot dishes, these neurons grow processes into compartments containing 25 ng/ml BDNF. Similar to the effects on Trk-mediated growth, this TrkB-mediated neuronal growth is dramatically reduced in a dose-dependent manner when the MEK inhibitor U0126 is added in conjunction with BDNF (FIG. 16).

We confirmed the requirement of MEK signaling for TrkB-mediated neuronal growth by coinfecting two adenoviruses, expressing DN MEK and TrkB, respectively. Neurons expressing both constructs exhibited greatly diminished neuronal growth in response to 25 ng/ml BDNF, as compared to neuronal growth mediated by TrkB expression alone.

### EXAMPLE 13

### A mutant TrkB Receptor Exhibiting Reduced Activation of MEK is Severely Deficient in Promoting Neuronal Growth

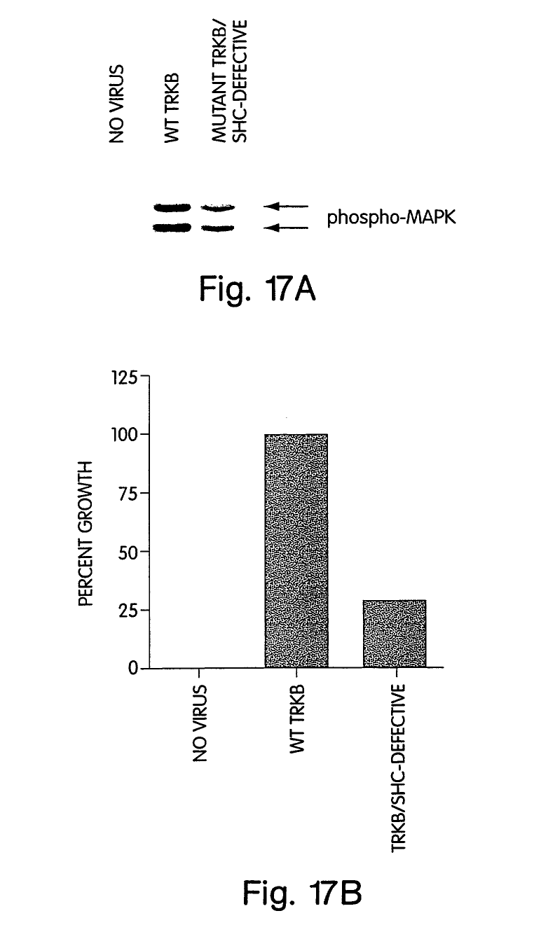

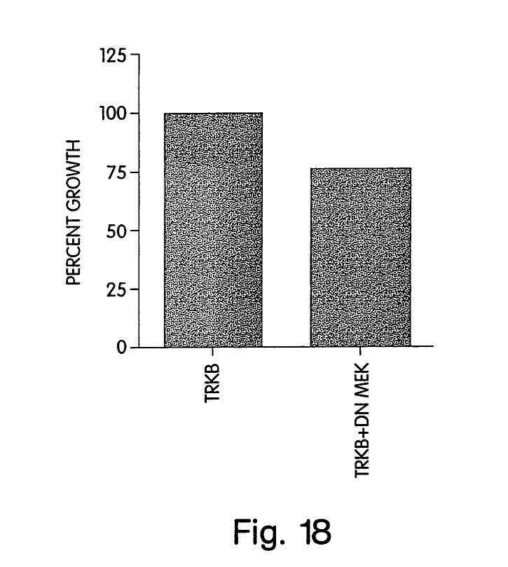

An advantage of using BDNF and exogenously delivered TrkB, rather than NGF and the endogenous TrkA found in sympathetic neurons, is that mutated or modified receptors can be introduced and tested without the complication of the presence of wild-type receptors. In one example, a TrkB receptor harboring a mutation in the SHC-association site was introduced in an adenoviral vector. Following addition of BDNF, this mutated receptor stimulated MEK only very weakly, as revealed by phosphorylation levels of MAPK (FIG. 17A) and neuronal growth (FIG. 17B). Thus, using such a strategy, one can identify mutant forms of TrkB that modulate neuronal growth without modulating neuronal survival. Such mutant forms may be useful in gene therapy.

### EXAMPLE 14

### Activity-Dependent Dendrite Formation in Sympathetic Neurons Neuronal Activity Causes Reversible Dendrite Formation in Sympathetic Neurons, and NGF Enhances this Effect

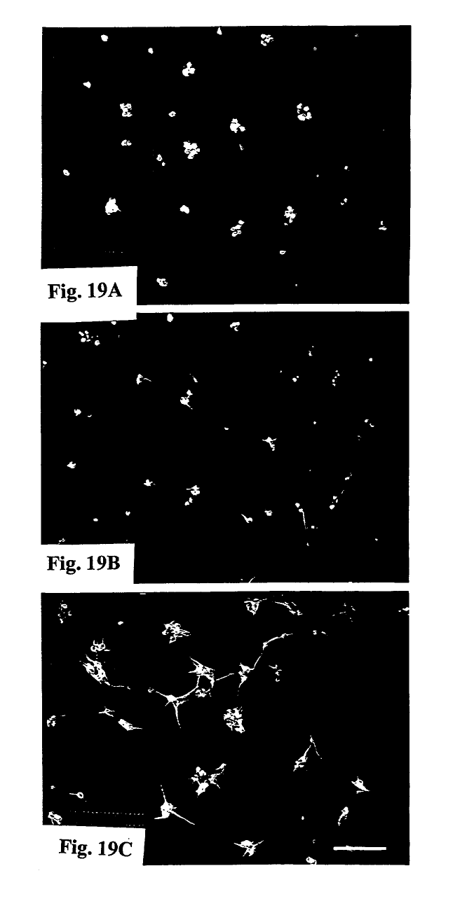

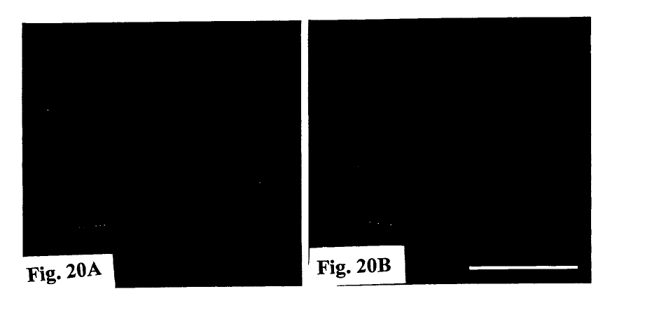

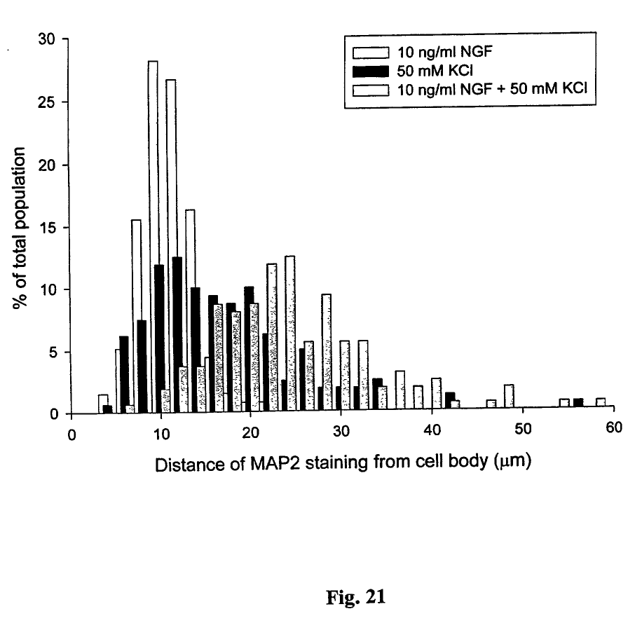

Neuronal depolarization causes dendrite formation in cultured sympathetic neurons, and NGF enhances this dendritogenesis. FIGS. 19A-19C, 20A, 20B show immunocytochemical analysis of sympathetic neurons cultured in the presence of NGF, switched to 10 ng/ml NGF, 50 mM KCl, or 10 ng/ml NGF+50 mM KCl for two days, and then stained for the dendrite-specific protein MAP2. FIGS. 20A and 20B are high magnification views of fields similar to those shown at lower-magnification in FIGS. 19A and 19C, respectively. The MAP2-immunoreactive processes show morphology characteristic of dendrites. The amount of dendritic formation could be readily quantified. Image analysis was used to measure the length of MAP2-positive dendrites on sympathetic neurons cultured in NGF, KCl, or NGF+KCl, and then these data were plotted as a distribution histogram (FIG. 21). Note that, while NGF on its own has little ability to cause dendritic growth, the addition of NGF to 50 mM KCl greatly enhances the KCl-dependent effect.

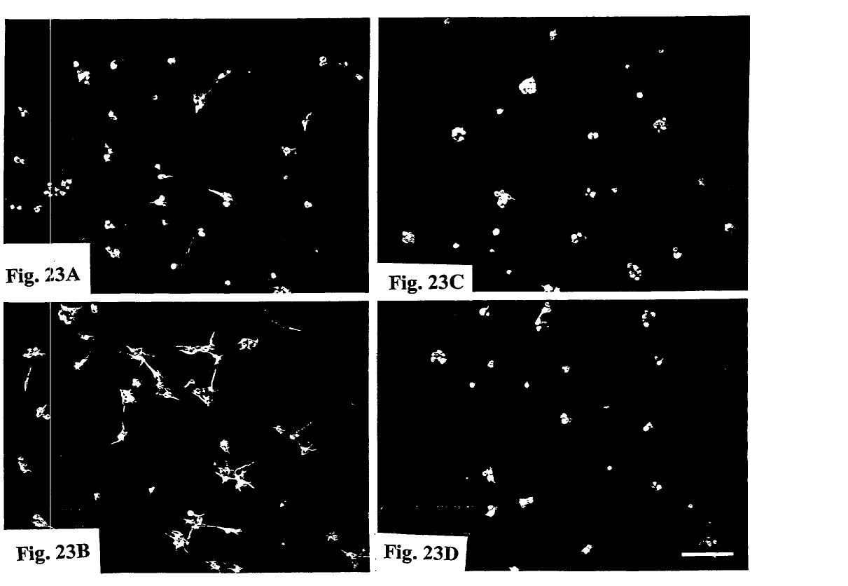

We found that activity-dependent dendrite formation is reversible. Immunocytochemical analysis of sympathetic neurons stained for MAP2 after being cultured in the presence of NGF, switched to 10 ng/ml NGF, 50 mM KCl, or NGF+KCl for three days, and then maintained in the same conditions or switched back to 10 ng/ml NGF for an additional two days indicated that, when neurons are maintained in depolarizing conditions, the MAP2-positive dendrites retract when KCl is removed (FIGS. 23A-23D).

Depolarization Promotes the Association of MAP2 with Microtubules, and Enhances Microtubule Stability

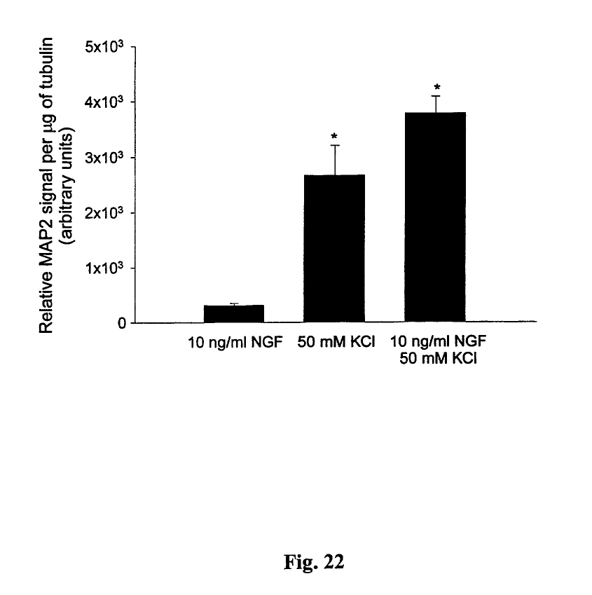

FIG. 22 shows quantitation of the amount of MAP2 associated with microtubules in sympathetic neurons switched into 10 ng/ml NGF, 50 mM KCl, or NGF+KCl for two days. Microtubules were isolated from cultured sympathetic neurons, and the relative amount of MAP2 present per microgram tubulin was determined using quantitative dot-blot analysis. Note that KCl greatly enhances the amount of MAP2 bound to microtubules, and that NGF enhances this effect to some degree. Depolarization enhances MAP2 levels and CamKII phosphorylation in sympathetic neurons. We performed western blot analysis of sympathetic neurons treated with 10 ng/ml NGF, 50 mM KCl, or NGF+KCl. Lysates containing equal amounts of protein were separated by SDS-PAGE, transferred to nitrocellulose, and then probed for MAP2, phosphorylated-CamKII, phosphorylated-ERKs, or total ERK protein. KCl increases the amount of MAP2 present in sympathetic neurons, and enhances CamKII phosphorylation. In contrast, 10 ng/ml NGF is a more potent stimulator of ERK phosphorylation than is 50 mM KCl.

Depolarization leads to CamKII phosphorylation in dendrites of sympathetic neurons. In order to verify that depolarization results in CamKII phosphorylation, we performed immunocytochemical analysis of sympathetic neurons switched into 10 ng/ml NGF, or 10 ng/ml NGF+50 mM KCl for two days. Neurons were double-labelled with antibodies specific to MAP2 and to phosphorylated-CamKII. In the presence of NGF alone, both MAP2 and phosphorylated-CamKII immunoreactivity are limited to the cell bodies of the cultured neurons. In contrast, when cultured in NGF+KCl, MAP2 is localized to dendrites and cell bodies, and phospho-CamKII immunostaining colocalizes in those dendrites. In addition, phospho-CamKII immunoreactivity is detectable in a particulate pattern along the length of neurites that are not dendrites.

Activation of Both CamKII and MEK are Essential for Activity-Dependent Dendritogenesis

Inhibition of CamKII or ERKs does not affect the survival of sympathetic neurons in NGF+KCl. We first established that the two Cam kinase inhibitors used in this study, KN-62 and AIP, do not affect ERK or Akt activation, that PD98059 does not affect CamKII activity, and that these drugs do effectively block the appropriate pathway under our conditions. We then performed MTT survival assays of sympathetic neurons switched to 10 ng/ml NGF, 50 mM KCl, or NGF+ plus KCl with or without the addition of KN-62 (inhibits CamKII), U0126 (inhibits MEK), or H-89 and forskolin (both of which perturb cAMP). None of these compounds perturbs sympathetic neurons cultured in the presence of NGF or NGF+KCl. In contrast, KN-62 dramatically reduces the survival of sympathetic neurons cultured only in 50 mM KCl.

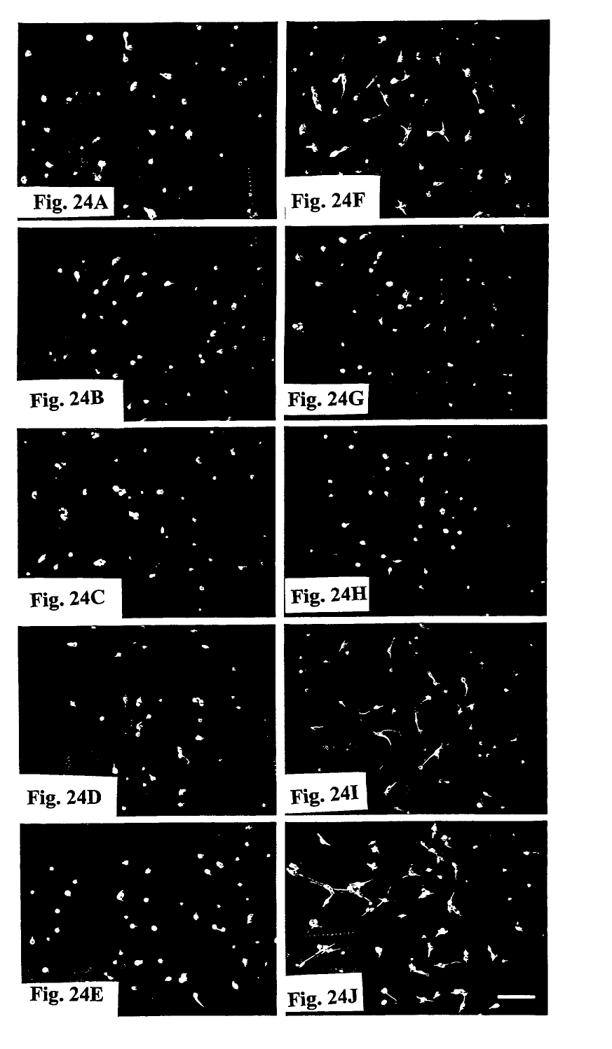

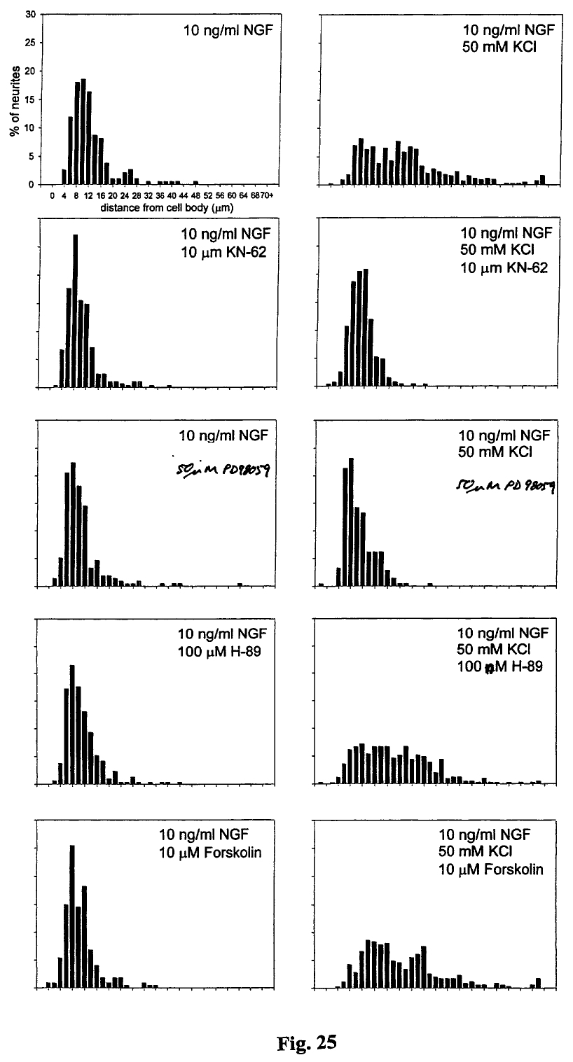

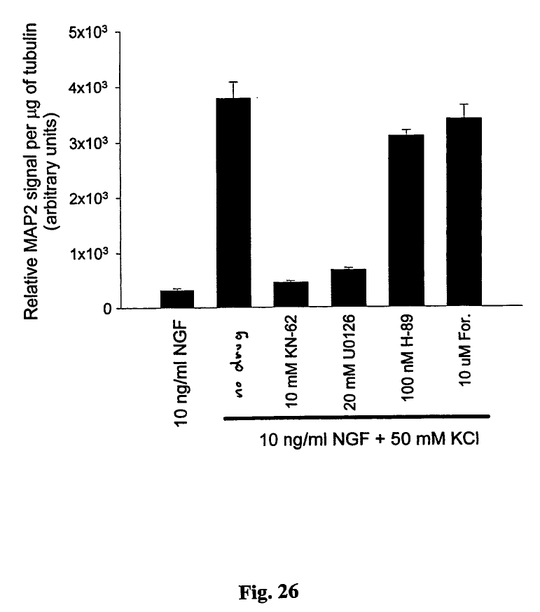

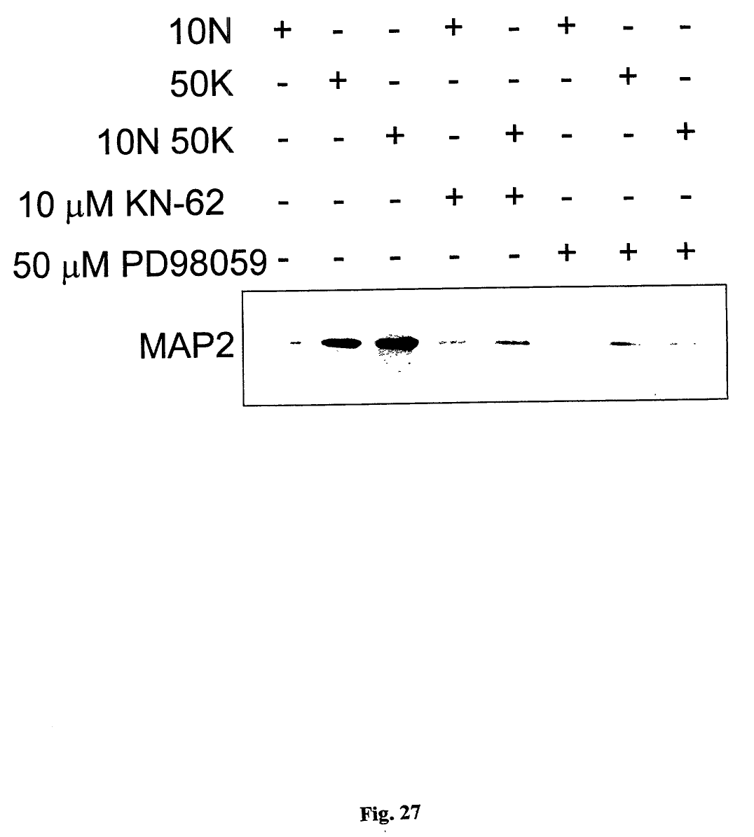

Inhibition of either CamKII or MEK is sufficient to inhibit activitydependent dendrite formation. Immunocytochemical analysis for MAP2 on sympathetic neurons cultured in the presence of NGF+KCl with or without 10 um KN62, 75 um PD98059, 100 nM H-89 or 10 uM forskolin. Inhibition of either CamKII or MEK completely abolished activity-dependent dendrite formation (FIGS. 24A-24J). Similar results were obtained with AIP (a second CamKII inhibitor) and U0129 (a second MEK inhibitor). These data are quantified in FIG. 25. Inhibition of CamKII or MEK also inhibited depolarization-induced association of MAP2 with microtubules, and the coincident increase in microtubule stability (FIG. 26). In this study, microtubule stability was determined in sympathetic neurons maintained in NGF or NGF+KCl, and treated with selective pharmacological inhibitors. Note that both KN62 and U0126 completely inhibit the KCl-induced increase in microtubule stability.

Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

What is claimed is:

1. A method for identifying a compound that increases or decreases neuritic growth, said method comprising the steps of:
(a) contacting a culture comprising neurons with a test compound; and
(b) measuring the amount of a protein or RNA that is preferentially found in the neurites of said neurons, wherein an increase in the amount of said protein or RNA relative to the amount of said protein or RNA in said culture not contacted with said test compound identifies said test compound as a compound that increases neuritic growth and a decrease in the amount of said protein or RNA relative to the amount of said protein or RNA in said culture not contacted with said test compound identifies said test compound as a compound that decreases neuritic growth.

2. The method of claim 1, wherein said protein is selected from the group consisting of alpha-tubulin, beta-tubulin, GAP-43, neurofilament light chain, neurofilament medium chain, neurofilament heavy chain, microtubule-associated protein (MAP) 1B, MAP4, MAP2C, and MAP2D.

3. The method of claim 1, wherein said neurons comprise sympathetic neurons, cholinergic neurons, sensory neurons, cortical neurons, motor neurons, and/or dorsal root ganglion neurons.

4. The method of claim 1, wherein said neurons comprise primary neurons, neurons derived from a cell line, and/or neurons derived from a stem cell.

5. The method of claim 1, wherein the test compound is selected from the group consisting of a peptide or protein, a carbohydrate, a lipid, a nucleic acid molecule, a natural organic molecule, and a synthetically derived organic molecule.

6. A method for identifying a compound that increases or decreases dendritic growth, said method comprising the steps of:
(a) contacting a culture comprising neurons with a test compound; and
(b) measuring the amount of a protein or RNA that is preferentially found in the dendrites of said neurons, wherein an increase in the amount of said protein or RNA relative to the amount of said protein or RNA in said culture not contacted with said test compound identifies said test compound as a compound that increases dendritic growth and a decrease in the amount of said protein or RNA relative to the amount of said protein or RNA in said culture not contacted with said test compound identifies said test compound as a compound that decreases dendritic growth.

7. The method of claim 6, wherein said protein is selected from the group consisting of MAP2A, MAP2B, dendrin, type II calcium-calmodulin-dependent protein (CamIIK), metabotropic glutamate receptor (mGluR) 1A, mGluR2, and type 1A serotonin receptor.

8. The method of claim 6, wherein said RNA is selected from the group consisting of MAP2A RNA, MAP2B RNA, dendrin RNA, and CamIIK RNA.

9. A method for identifying a compound that increases or decreases axonal growth, said method comprising the steps of:
(a) contacting a culture comprising neurons with a test compound; and
(b) measuring the amount of a protein or RNA that is preferentially found in the axons of said neurons, wherein an increase in the amount of said protein or RNA relative to the amount of said protein or RNA in said culture not contacted with said test compound identifies said test compound as a compound that increases axonal growth and a decrease in the amount of said protein or RNA relative to the amount of said protein or RNA in said culture not contacted with said test compound identifies said test compound as a compound that decreases axonal growth.

10. The method of claim 9, wherein said protein is selected from the group consisting of phosphorylated MAP1B, phosphorylated neurofilament heavy chain, and type 1B serotonin receptor.

11. A method of identifying a compound that increases or decreases neuronal growth, said method comprising the steps of:
(a) contacting a culture comprising neurons with NGF at levels sufficient to support cell survival;
(b) contacting said culture with a neuronal growth-inhibiting compound;
(c) contacting said culture with a test compound; and
(d) measuring the amount of a protein or RNA that is preferentially found in the axons, dendrites, or neurites of said neurons, wherein an increase in the amount of said protein or RNA relative to the amount of said protein or RNA in said culture not contacted with said test compound identifies said test compound as a compound that increases neuronal growth and a decrease in the amount of said protein or RNA relative to the amount of said protein or RNA in said culture not contacted with said test compound identifies said test compound as a compound that decreases neuronal growth.

12. The method of claim 11, wherein said neuronal growth-inhibiting compound is BDNF or an inhibitor of MEK signaling.

13. The method of claim 12, wherein said inhibitor of MEK signaling is a dominant-negative MEK.

14. The method of claim 12, wherein said inhibitor of MEK signaling is U0126 or PD98059.

15. A method of identifying a compound that increases or decreases neuronal growth, said method comprising the steps of:
(a) inhibiting MEK signaling in a culture comprising neurons;
(b) contacting said culture with a test compound; and
(c) measuring the amount of a protein or RNA that is preferentially found in the axons, dendrites, or neurites of said neurons, wherein an increase in the amount of said protein or RNA relative to the amount of said protein or RNA in said culture not contacted with said test compound identifies said test compound as a compound that increases neuronal growth and a decrease in the amount of said protein or RNA relative to the amount of said protein or RNA in said culture not contacted with said test compound identifies said test compound as a compound that decreases neuronal growth.

16. The method of claim 15, wherein said MEK signaling is inhibited by contacting said neuron with an inhibitor of MEK signaling.

17. The method of claim 16, wherein said inhibitor of MEK signaling is a dominant-negative MEK.

18. The method of claim 16, wherein said inhibitor of MEK signaling is U0126 or PD98059.

19. A method of identifying a compound that increases neuronal growth, said method comprising:
(a) activating p75^{NTR} signaling in a culture comprising neurons;
(b) contacting said culture with a test compound; and
(c) measuring the amount of a protein or RNA that is preferentially found in the axons, dendrites, or neurites of said neurons, wherein an increase in the amount of said protein or RNA relative to the amount of said protein or RNA in said culture not contacted with said test compound identifies said test compound as a compound that increases neuronal growth and a decrease in the amount of said protein or RNA relative to the amount of said protein or RNA in said culture not contacted with said test compound identifies said test compound as a compound that decreases neuronal growth.

20. The method of claim 19, wherein said p75^{NTR} signaling is activated by introducing ceramide into said neuron.
